(19) United States
(12) Patent Application Publication
(10) Pub. No.: US 2024/0158498 A1
(43) Pub. Date: May 16, 2024

(54) ANTI-SIGLEC-8 ANTIBODY FORMULATIONS

(71) Applicant: Allakos Inc., San Carlos, CA (US)

(72) Inventors: Jason WILLIAMS, San Carlos, CA (US); Nenad TOMASEVIC, Foster City, CA (US); Christopher Robert BEBBINGTON, San Mateo, CA (US)

(21) Appl. No.: 18/548,317

(22) PCT Filed: Mar. 2, 2022
(86) PCT No.: PCT/US2022/070927

§ 371 Date: Aug. 29, 2023

Related U.S. Application Data
(60) Provisional application No. 63/156,121, filed on Mar. 3, 2021.

(51) Int. Cl. C07K 16/28; A61K 47/02; A61K 47/12; A61K 47/18; A61K 47/26

(52) U.S. Cl. CPC C07K 16/2803; A61K 47/02; A61K 47/12; A61K 47/183; A61K 47/26

(57) ABSTRACT

The present disclosure provides formulations comprising an antibody that binds to a human Siglec-8, as well as articles of manufacture related thereto. In some embodiments, the formulations further comprise arginine, succinate, sodium chloride, and polysorbate.

### CROSS-REFERENCE TO RELATED APPLICATIONS

This application claims priority to U.S. Provisional Application No. 63/156,121, filed Mar. 3, 2021, the disclosures of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 701712000840SEQLIST.TXT, date recorded: Feb. 25, 2022, size: 32,868 bytes).

### FIELD OF THE INVENTION

The present disclosure relates to formulations and/or articles of manufacture comprising antibodies that bind to human Siglec-8.

### BACKGROUND

Siglecs (sialic acid-binding immunoglobulin-like lectins) are single-pass transmembrane cell surface proteins found predominantly on leukocytes and that are characterized by their specificity for sialic acids attached to cell-surface glycoconjugates. The Siglec family contains at least 15 members that are found in mammals (Pillai et al., Annu Rev Immunol., 2012, 30:357-392). These members include sialoadhesion (Siglec-1), CD22 (Siglec-2), CD33 (Siglec-3), myelin associated glycoprotein (Siglec-4), Siglec-5, OBBP1 (Siglec-6), AIRM1 (Siglec-7), SAF-2 (Siglec-8), and CD329 (Siglec-9). Siglec-8, a member that is expressed in humans but not in mouse, was first discovered as part of efforts to identify novel human eosinophil proteins. In addition to expression by eosinophils, it is also expressed by mast cells and basophils. Siglec-8 recognizes a sulfated glycan, i.e., 6′-sulfo-sialyl Lewis X or 6′-sulfo-sialyl-N-acetyl-S-lactosamine, and contains an intracellular immunoreceptor tyrosine-based inhibitory motif (ITIM) domain shown to inhibit mast cell function.

Along with mast cells, eosinophils can promote an inflammatory response that plays a beneficial functional role such as controlling an infection at a specific tissue site. During an inflammatory response, apoptosis of eosinophils can be inhibited through the activity of survival-promoting cytokines such as IL-3 and GM-CSF. However, an increase of activated eosinophils that are not rapidly removed by apoptosis can result in the release of eosinophil granule proteins at already inflamed sites which can damage tissue and cause inflammation to be further exacerbated. Several diseases have been shown to be linked to eosinophil activation such as Churg Strauss syndrome, rheumatoid arthritis, and allergic asthma (Wechsler et al., J Allergy Clin Immunol., 2012, 130(3):563-71). There is currently a need for therapies that can control the activity of immune cells involved in inflammation, such as the activity of eosinophils and mast cells.

Humanized antibodies that bind to Siglec-8 have been developed and are currently undergoing pre-clinical and clinical testing. See, e.g., U.S. Pat. No. 9,546,215. As such, there remains a need for formulations that allow for the storage, transportation, and administration of such antibodies while keeping them stable and preventing aggregation.

All references cited herein, including patent applications, patent publications, and scientific literature, are herein incorporated by reference in their entirety, as if each individual reference were specifically and individually indicated to be incorporated by reference.

### BRIEF SUMMARY

To meet this and other needs, the present disclosure relates, inter alia, to formulations comprising an antibody that binds to a human Siglec-8. Advantageously, these formulations provide improved stability and prevent antibody oligomerization and aggregation, e.g., due to agitation and/or freeze-thawing.

Accordingly, certain aspects of the present disclosure relate to formulations (e.g., liquid formulations) comprising (a) an antibody that binds to a human Siglec-8 in a concentration of about 5 mg/mL to about 15 mg/mL; (b) arginine in a concentration of about 50 mM to about 200 mM; (c) succinate in a concentration of about 5 mM to about 50 mM; (d) sodium chloride in a concentration of about 40 mM to about 150 mM; and (e) polysorbate in a concentration of about 0.002% to about 0.05%, wherein the formulation has a pH of about 5.0 to about 7.0 (e.g., a pH of about 6.0). In some embodiments, the antibody comprises: (1) a heavy chain variable region comprising: an HVR-H1 comprising the amino acid sequence of SEQ ID NO:1; an HVR-H2 comprising the amino acid sequence of SEQ ID NO:2; an HVR-H3 comprising the amino acid sequence of SEQ ID NO:3; and (1) a light chain variable region comprising: an HVR-L1 comprising the amino acid sequence of SEQ ID NO:4; an HVR-L2 comprising the amino acid sequence of SEQ ID NO:5; and an HVR-L3 comprising the amino acid sequence of SEQ ID NO:6.

In some embodiments, the antibody is in a concentration of about 15 mg/mL.

In some embodiments, the formulation comprises arginine, e.g., in a concentration of about 100 mM to about 200 mM. In some embodiments, the formulation comprises arginine in a concentration of about 100 mM to about 150 mM. In some embodiments, the formulation comprises arginine in a concentration of about 125 mM. In some embodiments, the arginine is an arginine HCl salt.

In some embodiments, the formulation comprises succinate, e.g., in a concentration of about 10 mM to about 50 mM. In some embodiments, the formulation comprises succinate in a concentration of about 10 mM to about 30 mM. In some embodiments, the formulation comprises succinate in a concentration of about 20 mM. In some embodiments, the succinate is a sodium succinate salt.

In some embodiments, the formulation comprises sodium chloride, e.g., in a concentration of about 50 mM to about 130 mM. In some embodiments, the formulation comprises sodium chloride in a concentration of about 75 mM to about 100 mM. In some embodiments, the formulation comprises sodium chloride in a concentration of about 80 mM.

In some embodiments, the formulation comprises polysorbate, e.g., in a concentration of about 0.01% to about 0.05%. In some embodiments, the formulation comprises polysorbate in a concentration of about 0.025%. In some embodiments, the polysorbate is polysorbate-80.

In some embodiments, the formulation comprises (a) the antibody in a concentration of 15 mg/mL; (b) arginine in a concentration of 125 mM; (c) succinate in a concentration of 20 mM; (d) sodium chloride in a concentration of 80 mM; and (e) polysorbate in a concentration of 0.025%, wherein the pH of the formulation is 6.0.

In some embodiments, less than 5% of the antibody in the formulation is aggregated after freezing and thawing, as measured by abundance of small antibody oligomers by size-exclusion chromatography high-performance liquid chromatography (SEC-HPLC). In some embodiments, less than 5% of the antibody in the formulation is aggregated after freezing and thawing five times, as measured by abundance of small antibody oligomers by size-exclusion chromatography high-performance liquid chromatography (SEC-HPLC). In some embodiments, less than 5% of the antibody in the formulation is aggregated after shaking overnight at 800 rpm, as measured by abundance of small antibody oligomers by size-exclusion chromatography high-performance liquid chromatography (SEC-HPLC). In some embodiments, after freezing and thawing the formulation has an absorbance at 400 nm (A400 nm) of less than about 150% of A400 nm of a reference standard, as measured by UV-Vis spectroscopy. In some embodiments, the formulation has an absorbance at 400 nm (A400 nm) of less than about 0.1 after freezing and thawing five times, as measured by UV-Vis spectroscopy. In some embodiments, the formulation has an absorbance at 400 nm (A400 nm) of less than about 0.1 after shaking overnight at 800 rpm, as measured by UV-Vis spectroscopy.

In some embodiments, the antibody comprises a Fc region and N-glycoside-linked carbohydrate chains linked to the Fc region, wherein less than 50% of the N-glycoside-linked carbohydrate chains of the antibody in the formulation contain a fucose residue. In some embodiments, substantially none of the N-glycoside-linked carbohydrate chains of the antibody in the composition contain a fucose residue.

In some embodiments, the antibody comprises a heavy chain variable region comprising the amino acid sequence of SEQ ID NO:7; and/or a light chain variable region comprising the amino acid sequence selected from SEQ ID NOs:8 or 9. In some embodiments, the antibody comprises a heavy chain Fc region comprising a human IgG Fc region. In some embodiments, the human IgG Fc region comprises a human IgG1. In some embodiments, the human IgG Fc region comprises a human IgG4. In some embodiments, the antibody comprises a heavy chain comprising the amino acid sequence of SEQ ID NO:19; and/or a light chain comprising the amino acid sequence selected from SEQ ID NOs:20 or 21. In some embodiments, the antibody comprises: (a) heavy chain variable region comprising: (1) an HC-FR1 comprising the amino acid sequence of SEQ ID NO:10; (2) an HVR-H1 comprising the amino acid sequence of SEQ ID NO:1; (3) an HC-FR2 comprising the amino acid sequence of SEQ ID NO:11; (4) an HVR-H2 comprising the amino acid sequence of SEQ ID NO:2; (5) an HC-FR3 comprising the amino acid sequence of SEQ ID NO:12; (6) an HVR-H3 comprising the amino acid sequence of SEQ ID NO:3; and (7) an HC-FR4 comprising the amino acid sequence of SEQ ID NOs:13; and/or (b) a light chain variable region comprising: (1) an LC-FR1 comprising the amino acid sequence of SEQ ID NO:14; (2) an HVR-L1 comprising the amino acid sequence of SEQ ID NO:4; (3) an LC-FR2 comprising the amino acid sequence of SEQ ID NO:15; (4) an HVR-L2 comprising the amino acid sequence of SEQ ID NO:5; (5) an LC-FR3 comprising the amino acid sequence of SEQ ID NO:16; (6) an HVR-L3 comprising the amino acid sequence of SEQ ID NO:6; and (7) an LC-FR4 comprising the amino acid sequence of SEQ ID NO:18. In some embodiments, the antibody comprises: (a) heavy chain variable region comprising: (1) an HC-FR1 comprising the amino acid sequence of SEQ ID NO:10; (2) an HVR-H1 comprising the amino acid sequence of SEQ ID NO:1; (3) an HC-FR2 comprising the amino acid sequence of SEQ ID NO:11; (4) an HVR-H2 comprising the amino acid sequence of SEQ ID NO:2; (5) an HC-FR3 comprising the amino acid sequence of SEQ ID NO:12; (6) an HVR-H3 comprising the amino acid sequence of SEQ ID NO:3; and (7) an HC-FR4 comprising the amino acid sequence of SEQ ID NOs:13; and/or (b) a light chain variable region comprising: (1) an LC-FR1 comprising the amino acid sequence of SEQ ID NO:14; (2) an HVR-L1 comprising the amino acid sequence of SEQ ID NO:4; (3) an LC-FR2 comprising the amino acid sequence of SEQ ID NO:15; (4) an HVR-L2 comprising the amino acid sequence of SEQ ID NO:5; (5) an LC-FR3 comprising the amino acid sequence of SEQ ID NO:17; (6) an HVR-L3 comprising the amino acid sequence of SEQ ID NO:6; and (7) an LC-FR4 comprising the amino acid sequence of SEQ ID NO:18. In some embodiments, the antibody is a monoclonal antibody. In some embodiments, the antibody is an IgG1 antibody. In some embodiments, the antibody has been engineered to improve antibody-dependent cell-mediated cytotoxicity (ADCC) activity. In some embodiments, the antibody comprises at least one amino acid substitution in the Fc region that improves ADCC activity. In some embodiments, at least one or two of the heavy chains of the antibody is non-fucosylated. In some embodiments, the antibody is a human antibody, a humanized antibody or a chimeric antibody. In some embodiments, the antibody comprises an antibody fragment selected from the group consisting of Fab, Fab′-SH, Fv, scFv, and (Fab′)₂ fragments.

In some embodiments, the antibody comprises a Fc region and N-glycoside-linked carbohydrate chains linked to the Fc region, wherein less than 50% of the N-glycoside-linked carbohydrate chains of the antibody in the composition contain a fucose residue. In some embodiments, substantially none of the N-glycoside-linked carbohydrate chains of the antibody in the composition contain a fucose residue. In some embodiments, the antibody comprises a heavy chain Fc region comprising a human IgG Fc region. In some embodiments, the human IgG Fc region comprises a human IgG1 Fc region. In some embodiments, the human IgG1 Fc region is non-fucosylated. In some embodiments, the human IgG Fc region comprises a human IgG4 Fc region. In some embodiments, the human IgG4 Fc region comprises the amino acid substitution S228P, wherein the amino acid residues are numbered according to the EU index as in Kabat. In some embodiments, the antibody depletes blood eosinophils and/or inhibits mast cell activation. In some embodiments, the antibody is a monoclonal antibody.

In another aspect, the present disclosure provides articles of manufacture or kits comprising a container enclosing the formulation according to any of the above embodiments. In some embodiments, the container is a glass vial. In some embodiments, the article of manufacture or kit further comprises instructions for administering the formulation intravenously (e.g., via intravenous infusion) or subcutaneously (e.g., via subcutaneous injection).

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIG. 1A shows the binding of antibody HEKA as part of the indicated anti-Siglec-8 antibody formulations to the Siglec-8 extracellular domain (ECD) at time 0, as measured by ELISA. EC50 values for each formulation are indicated.

FIG. 1B shows the binding of antibody HEKF as part of the indicated anti-Siglec-8 antibody formulations to the Siglec-8 extracellular domain (ECD) at time 0, as measured by ELISA. EC50 values for each formulation are indicated.

FIG. 1C shows the binding of antibody HEKA as part of the indicated anti-Siglec-8 antibody formulations after 1 week at 37° C. to the Siglec-8 extracellular domain (ECD), as measured by ELISA. EC50 values for each formulation are indicated.

FIG. 1D shows the binding of antibody HEKF as part of the indicated anti-Siglec-8 antibody formulations after 1 week at 37° C. to the Siglec-8 extracellular domain (ECD), as measured by ELISA. EC50 values for each formulation are indicated.

FIGS. 2A & 2B show the results of UV-Vis spectroscopic analysis of the indicated anti-Siglec-8 antibody formulations. Each formulation was assayed after 2 weeks at 4° C. (formulation #0.4), 2 weeks at 25° C. (formulation #0.25), 2 weeks at 37° C. (formulation #0.37), or after freeze-thawing for 1 cycle (formulation #.FT) or 5 cycles (formulation #.FT5x). Shown are the absorbance values at 400 nm (A400 nm) for antibodies HEKA (FIG. 2A) and HEKF (FIG. 2B).

FIGS. 3A-3C show the results of size-exclusion chromatography high-performance liquid chromatography (SEC-HPLC) analysis of anti-Siglec-8 antibody formulations. FIG. 3A shows the SEC-HPLC peaks of molecular weight standards, reference HEKA and HEKF antibodies, and dextran. FIG. 3B shows the SEC-HPLC profile of antibody HEKA formulation in pH 5 buffer (see Table A) after storage for 2 weeks at the indicated temperature, as compared to reference. FIG. 3C shows the SEC-HPLC profile of antibody HEKF formulation in pH 5 buffer (see Table A) after storage for 2 weeks at the indicated temperature, as compared to reference. Smaller peaks indicative of antibody small oligomers are labeled in FIGS. 3B & 3C.

FIGS. 4A & 4B show the percentage of antibody oligomers formed in the indicated anti-Siglec-8 antibody formulations, as assayed by size-exclusion chromatography high-performance liquid chromatography (SEC-HPLC) after 2 weeks. Shown are the results for antibodies HEKA (FIG. 4A) and HEKF (FIG. 4B). Each formulation was assayed after 2 weeks at 4° C. (formulation #0.4), 2 weeks at 25° C. (formulation #0.25), 2 weeks at 37° C. (formulation #0.37), or after freeze-thawing for 1 cycle (formulation #.FT) or 5 cycles (formulation #.FT5x).

FIGS. 5A-5E show the results of SDS-PAGE analysis of HEKA and HEKF antibodies after storage at the indicated temperature in the indicated formulations. FIG. 5A shows the results of reducing and non-reducing SDS-PAGE analysis of HEKA and HEKF at time 0. FIGS. 5B & 5C show the results of reducing/non-reducing SDS-PAGE analysis of HEKA and HEKF, respectively, after 1 week. FIGS. 5D & 5E show the results of reducing/non-reducing SDS-PAGE analysis of HEKA and HEKF, respectively, after 2 weeks.

FIGS. 6A & 6B show the results of UV-Vis spectroscopic analysis of the effect of arginine concentration in the indicated anti-Siglec-8 antibody formulations. Each formulation was assayed (by measuring A400 nm) after freeze-thawing for 1 cycle (middle bar) or 5 cycles (rightmost bar), and compared against a corresponding antibody formulation not subjected to freeze-thaw (leftmost bar). Shown are the results for antibodies HEKA (FIG. 6A) and HEKF (FIG. 6B).

FIGS. 7A & 7B show the results of size-exclusion chromatography high-performance liquid chromatography (SEC-HPLC) analysis of anti-Siglec-8 antibody formulations. FIG. 7A shows the SEC-HPLC profile of antibody HEKA formulation in a formulation lacking arginine (see buffer 1 in Table E) after one or five freeze-thaw cycles, as compared to reference. FIG. 7B shows the SEC-HPLC profile of antibody HEKF formulation in a formulation lacking arginine (see buffer 1 in Table E) after one or five freeze-thaw cycles, as compared to reference. Smaller peaks indicative of antibody small oligomers are labeled.

FIGS. 8A & 8B show the results of size-exclusion chromatography high-performance liquid chromatography (SEC-HPLC) analysis of anti-Siglec-8 antibody formulations. FIG. 8A shows the percentage of small oligomers as measured by SEC-HPLC of the indicated formulations with antibody HEKA after 1 (formulation 4.1 FT) or 5 (formulation 4.5×) freeze-thaw cycles, as compared with antibody formulation not subjected to freeze-thaw. FIG. 8B shows the percentage of small oligomers as measured by SEC-HPLC of the indicated formulations with antibody HEKF after 1 (4.1 FT) or 5 (#0.5×) freeze-thaw cycles, as compared with antibody formulation not subjected to freeze-thaw.

FIG. 9 shows the results of UV-Vis spectroscopic analysis (A400 nm) of HEKA antibody formulations with the indicated concentration of polysorbate-80 after 2 (left bar) or 4 (right bar) days of agitation.

FIG. 10 shows the proportion of oligomerization of HEKA antibody formulations with the indicated concentration of polysorbate-80, as measured by SEC-HPLC.

FIG. 11 shows the effect of formulation composition on HEKA antibody aggregation after freeze-thaw, as measured by absorbance at 400 nm.

FIG. 12A shows the effect of formulation composition on HEKA antibody aggregation after agitation for 1 day at 800 rpm, as measured by absorbance at 400 nm. Formulations are described in Table G.

FIG. 12B shows the effect of formulation composition on HEKA antibody oligomer formation after agitation for 1 day at 800 rpm, as measured by SEC-HPLC. Formulations are described in Table G.

### DETAILED DESCRIPTION

### I. Definitions

It is to be understood that the present disclosure is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

The term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)2, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ ands isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, CT, 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in the present disclosure. Common allotypic variants in human populations are those designated by the letters a, f, n, z.

An “isolated” antibody is one that has been identified, separated and/or recovered from a component of its production environment (e.g., naturally or recombinantly). In some embodiments, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In some embodiments, the polypeptide is purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated polypeptide or antibody is prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain. In some embodiments, monoclonal antibodies are highly specific, being directed against a single antigenic site. In some embodiments, monoclonal antibodies are highly specific, being directed against multiple antigenic sites (such as a bispecific antibody or a multispecific antibody). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method, recombinant DNA methods, phage-display technologies, and technologies for producing human or human-like antibodies in animals that have parts or all of the human immunoglobulin loci or genes encoding human immunoglobulin sequences.

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. In some embodiments, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the present disclosure comprise a portion of an intact antibody, generally including the antigen binding or variable region of the intact antibody or the Fv region of an antibody which retains or has modified FcR binding capability. Examples of antibody fragments include linear antibody, single-chain antibody molecules and multispecific antibodies formed from antibody fragments.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used as a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. In some embodiments, the number of these amino acid substitutions in the FR are no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. In some embodiments, humanized antibodies are directed against a single antigenic site. In some embodiments, humanized antibodies are directed against multiple antigenic sites. An alternative humanization method is described in U.S. Pat. No. 7,981,843 and U.S. Patent Application Publication No. 2006/0134098.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N J, 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5th Ed. Public Health Service, National Institute of Health, Bethesda, MD (1991)). Chothia HVRs refer instead to the location of the structural loops (Chothia and Lesk J. Mol. Biol. 196:901-917 (1987)). The “contact” HVRs are based on an analysis of the available complex crystal structures. The residues from each of these I-WRs are noted below.

| Loop | Kabat | Chothia | Contact |
|---|---|---|---|
| L1 | L24-L34 | L26-L34 | L30-L36 |
| L2 | L50-L56 | L50-L56 | L46-L55 |
| L3 | L89-L97 | L91-L96 | L89-L96 |
| H1 | H31-H35B | H26-H32 | H30-H35B (Kabat Numbering) |
| H1 | H31-H35 | H26-H32 | H30-H35 (Chothia Numbering) |
| H2 | H50-H65 | H53-H56 | H47-H58 |
| H3 | H95-H102 | H95-H102 | H93-H101 |

Unless otherwise indicated, the variable-domain residues (HVR residues and framework region residues) are numbered according to Kabat et al., supra.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

An “acceptor human framework” for the purposes herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

An antibody that “binds to”, “specifically binds to” or is “specific for” a particular a polypeptide or an epitope on a particular polypeptide is one that binds to that particular polypeptide or epitope on a particular polypeptide without substantially binding to any other polypeptide or polypeptide epitope. In some embodiments, binding of an anti-Siglec-8 antibody described herein (e.g., an antibody that binds to human Siglec-8) to an unrelated non-Siglec-8 polypeptide is less than about 10% of the antibody binding to Siglec-8 as measured by methods known in the art (e.g., enzyme-linked immunosorbent assay (ELISA)). In some embodiments, an antibody that binds to a Siglec-8 (e.g., an antibody that binds to human Siglec-8) has a dissociation constant (Kd) of ≤100 nM, ≤10 nM, ≤2 nM, ≤1 nM, ≤0.7 nM, ≤0.6 nM, ≤0.5 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

The term “anti-Siglec-8 antibody” or “an antibody that binds to human Siglec-8” refers to an antibody that binds to a polypeptide or an epitope of human Siglec-8 without substantially binding to any other polypeptide or epitope of an unrelated non-Siglec-8 polypeptide.

The term “Siglec-8” as used herein refers to a human Siglec-8 protein. The term also includes naturally occurring variants of Siglec-8, including splice variants or allelic variants. The amino acid sequence of an exemplary human Siglec-8 is shown in SEQ ID NO:25. The amino acid sequence of another exemplary human Siglec-8 is shown in SEQ ID NO:26. In some embodiments, a human Siglec-8 protein comprises the human Siglec-8 extracellular domain fused to an immunoglobulin Fc region.

Human Siglec-8 Amino Acid Sequence
(SEQ ID NO: 25)
GYLLQVQELVTVQEGLCVHVPCSFSYPQDGWTDSDPVHGYWFRAGDRPYQDAPVATN
NPDREVQAETQGRFQLLGDIWSNDCSLSIRDARKRDKGSYFFRLERGSMKWSYKSQLN
YKTKQLSVFVTALTHRPDILILGTLESGHSRNLTCSVPWACKQGTPPMISWIGASVSSPG
PTTARSSVLTLTPKPQDHGTSLTCQVTLPGTGVTTTSTVRLDVSYPPWNLTMTVFQGDA
TASTALGNGSSLSVLEGQSLRLVCAVNSNPPARLSWTRGSLTLCPSRSSNPGLLELPRVH
VRDEGEFTCRAQNAQGSQHISLSLSLQNEGTGTSRPVSQVTLAAVGGAGATALAFLSFC
IIFIIVRSCRKKSARPAAGVGDTGMEDAKAIRGSASQGPLTESWKDGNPLKKPPPAVAPS
SGEEGELHYATLSFHKVKPQDPQGQEATDSEYSEIKIHKRETAETQACLRNHNPSSKEV
RG
Human Siglec-8 Amino Acid Sequence
(SEQ ID NO: 26)
GYLLQVQELVTVQEGLCVHVPCSFSYPQDGWTDSDPVHGYWFRAGDRPYQDAPVATN
NPDREVQAETQGRFQLLGDIWSNDCSLSIRDARKRDKGSYFFRLERGSMKWSYKSQLN
YKTKQLSVFVTALTHRPDILILGTLESGHPRNLTCSVPWACKQGTPPMISWIGASVSSPG
PTTARSSVLTLTPKPQDHGTSLTCQVTLPGTGVTTTSTVRLDVSYPPWNLTMTVFQGDA
TASTALGNGSSLSVLEGQSLRLVCAVNSNPPARLSWTRGSLTLCPSRSSNPGLLELPRVH
VRDEGEFTCRAQNAQGSQHISLSLSLQNEGTGTSRPVSQVTLAAVGGAGATALAFLSFC
IIFIIVRSCRKKSARPAAGVGDTGMEDAKAIRGSASQGPLTESWKDGNPLKKPPPAVAPS
SGEEGELHYATLSFHKVKPQDPQGQEATDSEYSEIKIHKRETAETQACLRNHNPSSKEV
RG

Antibodies that “induce apoptosis” or are “apoptotic” are those that induce programmed cell death as determined by standard apoptosis assays, such as binding of annexin V, fragmentation of DNA, cell shrinkage, dilation of endoplasmic reticulum, cell fragmentation, and/or formation of membrane vesicles (called apoptotic bodies). For example, the apoptotic activity of the anti-Siglec-8 antibodies (e.g., an antibody that binds to human Siglec-8) of the present disclosure can be shown by staining cells with annexin V.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). In some embodiments, an anti-Siglec-8 antibody (e.g., an antibody that binds to human Siglec-8) described herein enhances ADCC. To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998). Other Fc variants that alter ADCC activity and other antibody properties include those disclosed by Ghetie et al., Nat Biotech. 15:637-40, 1997; Duncan et al, Nature 332:563-564, 1988; Lund et al., J. Immunol 147:2657-2662, 1991; Lund et al, Mol Immunol 29:53-59, 1992; Alegre et al, Transplantation 57:1537-1543, 1994; Hutchins et al., Proc Natl. Acad Sci USA 92:11980-11984, 1995; Jefferis et al, Immunol Lett. 44:111-117, 1995; Lund et al., FASEB J9:115-119, 1995; Jefferis et al, Immunol Lett 54:101-104, 1996; Lund et al, J Immunol 157:4963-4969, 1996; Armour et al., Eur J Immunol 29:2613-2624, 1999; Idusogie et al, J Immunol 164:4178-4184, 200; Reddy et al, J Immunol 164:1925-1933, 2000; Xu et al., Cell Immunol 200:16-26, 2000; Idusogie et al, J Immunol 166:2571-2575, 2001; Shields et al., J Biol Chem 276:6591-6604, 2001; Jefferis et al, Immunol Lett 82:57-65. 2002; Presta et al., Biochem Soc Trans 30:487-490, 2002; Lazar et al., Proc. Natl. Acad. Sci. USA 103:4005-4010, 2006; U.S. Pat. Nos. 5,624,821; 5,885,573; 5,677,425; 6,165,745; 6,277,375; 5,869,046; 6,121,022; 5,624,821; 5,648,260; 6,194,551; 6,737,056; 6,821,505; 6,277,375; 7,335,742; and 7,317,091.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present disclosure include human IgG1, IgG2, IgG3 and IgG4. A single amino acid substitution (S228P according to Kabat numbering; designated IgG4Pro) may be introduced to abolish the heterogeneity observed in recombinant IgG4 antibody. See Angal, S. et al. (1993) Mol Immunol 30, 105-108.

“Non-fucosylated” or “fucose-deficient” antibody refers to a glycosylation antibody variant comprising an Fc region wherein a carbohydrate structure attached to the Fc region has reduced fucose or lacks fucose. In some embodiments, an antibody with reduced fucose or lacking fucose has improved ADCC function. Non-fucosylated or fucose-deficient antibodies have reduced fucose relative to the amount of fucose on the same antibody produced in a cell line. In some embodiments, a non-fucosylated or fucose-deficient antibody composition contemplated herein is a composition wherein less than about 50% of the N-linked glycans attached to the Fc region of the antibodies in the composition comprise fucose.

The terms “fucosylation” or “fucosylated” refers to the presence of fucose residues within the oligosaccharides attached to the peptide backbone of an antibody. Specifically, a fucosylated antibody comprises a (1,6)-linked fucose at the innermost N-acetylglucosamine (GlcNAc) residue in one or both of the N-linked oligosaccharides attached to the antibody Fc region, e.g. at position Asn 297 of the human IgG1 Fc domain (EU numbering of Fc region residues). Asn297 may also be located about +3 amino acids upstream or downstream of position 297, i.e. between positions 294 and 300, due to minor sequence variations in immunoglobulins.

The “degree of fucosylation” is the percentage of fucosylated oligosaccharides relative to all oligosaccharides identified by methods known in the art e.g., in an N-glycosidase F treated antibody composition assessed by matrix-assisted laser desorption-ionization time-of-flight mass spectrometry (MALDI-TOF MS). In a composition of a “fully fucosylated antibody” essentially all oligosaccharides comprise fucose residues, i.e. are fucosylated. In some embodiments, a composition of a fully fucosylated antibody has a degree of fucosylation of at least about 90%. Accordingly, an individual antibody in such a composition typically comprises fucose residues in each of the two N-linked oligosaccharides in the Fc region. Conversely, in a composition of a “fully non-fucosylated” antibody essentially none of the oligosaccharides are fucosylated, and an individual antibody in such a composition does not contain fucose residues in either of the two N-linked oligosaccharides in the Fc region. In some embodiments, a composition of a fully non-fucosylated antibody has a degree of fucosylation of less than about 10%. In a composition of a “partially fucosylated antibody” only part of the oligosaccharides comprise fucose. An individual antibody in such a composition can comprise fucose residues in none, one or both of the N-linked oligosaccharides in the Fc region, provided that the composition does not comprise essentially all individual antibodies that lack fucose residues in the N-linked oligosaccharides in the Fc region, nor essentially all individual antibodies that contain fucose residues in both of the N-linked oligosaccharides in the Fc region. In one embodiment, a composition of a partially fucosylated antibody has a degree of fucosylation of about 10% to about 80% (e.g., about 50% to about 80%, about 60% to about 80%, or about 70% to about 80%).

“Binding affinity” as used herein refers to the strength of the non-covalent interactions between a single binding site of a molecule (e.g., an antibody) and its binding partner (e.g., an antigen). In some embodiments, the binding affinity of an antibody for a Siglec-8 (which may be a dimer, such as the Siglec-8-Fc fusion protein described herein) can generally be represented by a dissociation constant (Kd). Affinity can be measured by common methods known in the art, including those described herein.

“Binding avidity” as used herein refers to the binding strength of multiple binding sites of a molecule (e.g., an antibody) and its binding partner (e.g., an antigen).

An “isolated” nucleic acid molecule encoding the antibodies herein is a nucleic acid molecule that is identified and separated from at least one contaminant nucleic acid molecule with which it is ordinarily associated in the environment in which it was produced. In some embodiments, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “pharmaceutical formulation” (or, alternatively, “formulation”) refers to a preparation that is in such form as to permit the biological activity of the active ingredient to be effective, and that contains no additional components that are unacceptably toxic to an individual to which the formulation would be administered. Such formulations are sterile.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “treatment” or “treating” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease (e.g., a Siglec-8-associated disease or disorder) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

As used herein, “in conjunction with” or “in combination with” refers to administration of one treatment modality in addition to another treatment modality. As such, “in conjunction with” or “in combination with” refers to administration of one treatment modality before, during or after administration of the other treatment modality to the individual.

As used herein, the term “prevention” or “preventing” includes providing prophylaxis with respect to occurrence or recurrence of a disease in an individual. An individual may be predisposed to a disease, susceptible to a disease, or at risk of developing a disease, but has not yet been diagnosed with the disease. In some embodiments, anti-Siglec-8 antibodies (e.g., an antibody that binds to human Siglec-8) described herein are used to delay development of a disease (e.g., a Siglec-8-associated disease or disorder).

As used herein, an individual “at risk” of developing a disease (e.g., a Siglec-8-associated disease or disorder) may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of the disease, as known in the art. An individual having one or more of these risk factors has a higher probability of developing the disease than an individual without one or more of these risk factors.

An “effective amount” refers to at least an amount effective, at dosages and for periods of time necessary, to achieve the desired or indicated effect, including a therapeutic or prophylactic result. An effective amount can be provided in one or more administrations. A “therapeutically effective amount” is at least the minimum concentration required to effect a measurable improvement of a particular disease. A therapeutically effective amount herein may vary according to factors such as the disease state, age, sex, and weight of the patient, and the ability of the antibody to elicit a desired response in the individual. A therapeutically effective amount may also be one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in individuals prior to or at the earlier stage of disease, the prophylactically effective amount can be less than the therapeutically effective amount.

“Chronic” administration refers to administration of the medicament(s) in a continuous as opposed to acute mode, so as to maintain the initial therapeutic effect (activity) for an extended period of time. “Intermittent” administration is treatment that is not consecutively done without interruption, but rather is cyclic in nature.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is a human.

### II. Anti-Siglec-8 Antibody Formulations

Provided herein are formulations (e.g., pharmaceutical formulations) comprising any of the anti-Siglec-8 antibodies described herein (e.g., an antibody that binds to Siglec-8). In some embodiments, the formulation is a liquid formulation. In some embodiments, the liquid formulation is stored at between about 2° C. and about 8° C. Advantageously, these formulations were found to improve stability and reduce aggregation and oligomerization (e.g., after agitation or freeze-thawing) of antibodies of the present disclosure.

A. Anti-Siglec-8 Antibodies

Any of the anti-Siglec-8 antibodies described herein may find use in a formulation of the present disclosure (see, e.g., section D below). In some embodiments, the antibody is present in a formulation of the present disclosure in an amount or concentration of between about 5 mg/mL and about 15 mg/mL, or between about 10 mg/mL and about 15 mg/mL. For example, in some embodiments, the antibody is present at about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, or about 15 mg/mL.

For example, in some embodiments, the antibody comprises: (1) a heavy chain variable region comprising: an HVR-H1 comprising the amino acid sequence of SEQ ID NO:1; an HVR-H2 comprising the amino acid sequence of SEQ ID NO:2; an HVR-H3 comprising the amino acid sequence of SEQ ID NO:3; and (1) a light chain variable region comprising: an HVR-L1 comprising the amino acid sequence of SEQ ID NO:4; an HVR-L2 comprising the amino acid sequence of SEQ ID NO:5; and an HVR-L3 comprising the amino acid sequence of SEQ ID NO:6.

In some embodiments, the antibody comprises: (1) a heavy chain variable region comprising: an HC-FR1 comprising the amino acid sequence of SEQ ID NO:10; an HVR-H1 comprising the amino acid sequence of SEQ ID NO:1; an HC-FR2 comprising the amino acid sequence of SEQ ID NO:11; an HVR-H2 comprising the amino acid sequence of SEQ ID NO:2; an HC-FR3 comprising the amino acid sequence of SEQ ID NO:12; an HVR-H3 comprising the amino acid sequence of SEQ ID NO:3; and an HC-FR4 comprising the amino acid sequence of SEQ ID NOs:13; and (1) a light chain variable region comprising: an LC-FR1 comprising the amino acid sequence of SEQ ID NO:14; an HVR-L1 comprising the amino acid sequence of SEQ ID NO:4; an LC-FR2 comprising the amino acid sequence of SEQ ID NO:15; an HVR-L2 comprising the amino acid sequence of SEQ ID NO:5; an LC-FR3 comprising the amino acid sequence of SEQ ID NO:16; an HVR-L3 comprising the amino acid sequence of SEQ ID NO:6; and an LC-FR4 comprising the amino acid sequence of SEQ ID NO:18. Additional descriptions of such antibodies are provided, e.g., in U.S. Pat. No. 9,546,215.

In some embodiments, the antibody comprises: (1) a heavy chain variable region comprising: an HC-FR1 comprising the amino acid sequence of SEQ ID NO:10; an HVR-H1 comprising the amino acid sequence of SEQ ID NO:1; an HC-FR2 comprising the amino acid sequence of SEQ ID NO:11; an HVR-H2 comprising the amino acid sequence of SEQ ID NO:2; an HC-FR3 comprising the amino acid sequence of SEQ ID NO:12; an HVR-H3 comprising the amino acid sequence of SEQ ID NO:3; and an HC-FR4 comprising the amino acid sequence of SEQ ID NOs:13; and (1) a light chain variable region comprising: an LC-FR1 comprising the amino acid sequence of SEQ ID NO:14; an HVR-L1 comprising the amino acid sequence of SEQ ID NO:4; an LC-FR2 comprising the amino acid sequence of SEQ ID NO:15; an HVR-L2 comprising the amino acid sequence of SEQ ID NO:5; an LC-FR3 comprising the amino acid sequence of SEQ ID NO:17; an HVR-L3 comprising the amino acid sequence of SEQ ID NO:6; and an LC-FR4 comprising the amino acid sequence of SEQ ID NO:18.

In some embodiments, the heavy chain variable region comprises the amino acid sequence of SEQ ID NO:7. In some embodiments, the light chain variable region comprises the amino acid sequence of SEQ ID NO:8 or SEQ ID NO:21. For example, in some embodiments, the antibody comprises a heavy chain that comprises a heavy chain variable region comprising the amino acid sequence of SEQ ID NO:7 and a light chain that comprises a light chain variable region comprising the amino acid sequence of SEQ ID NO:8. In other embodiments, the antibody comprises a heavy chain that comprises a heavy chain variable region comprising the amino acid sequence of SEQ ID NO:7 and a light chain that comprises a light chain variable region comprising the amino acid sequence of SEQ ID NO:21.

In some embodiments, the antibody is a human antibody, a humanized antibody, or a chimeric antibody. In some embodiments, the antibody depletes blood eosinophils and inhibits mast cell activation.

In one aspect, an anti-Siglec-8 antibody described herein is a monoclonal antibody. In one aspect, an anti-Siglec-8 antibody described herein is an antibody fragment (including antigen-binding fragment), e.g., a Fab, Fab′-SH, Fv, scFv, or (Fab′)₂ fragment. In one aspect, an anti-Siglec-8 antibody described herein comprises an antibody fragment (including antigen-binding fragment), e.g., a Fab, Fab′-SH, Fv, scFv, or (Fab′)₂ fragment. In one aspect, an anti-Siglec-8 antibody described herein is a chimeric, humanized, or human antibody. In one aspect, any of the anti-Siglec-8 antibodies described herein are purified.

In some embodiments, the antibody comprises a heavy chain Fc region comprising a human IgG Fc region, including but not limited to a human IgG1 or a human IgG4 Fc region.

In some embodiments, the Fc region comprises one or more mutations, e.g., as compared to a wild-type human Fc region. For example, in some embodiments, the Fc region is a human IgG4 Fc region comprising an S228P substitution (amino acid residue numbering according to EU index as in Kabat).

In some embodiments, the antibody comprises a heavy chain that comprises the amino acid sequence of SEQ ID NO:19 and a light chain that comprises the amino acid sequence of SEQ ID NO:20. In other embodiments, the antibody comprises a heavy chain that comprises the amino acid sequence of SEQ ID NO:19 and a light chain that comprises the amino acid sequence of SEQ ID NO:21. In other embodiments, the antibody comprises a heavy chain that comprises the amino acid sequence of SEQ ID NO:27 and a light chain that comprises the amino acid sequence of SEQ ID NO:20.

In some embodiments, the antibody has been engineered to improve antibody-dependent cell-mediated cytotoxicity (ADCC) activity.

In some aspects, provided herein is a formulation comprising an anti-Siglec-8 antibody described herein, wherein the antibody comprises a Fc region and N-glycoside-linked carbohydrate chains linked to the Fc region, wherein less than about 50% of the N-glycoside-linked carbohydrate chains contain a fucose residue. In some embodiments, the antibody comprises a Fc region and N-glycoside-linked carbohydrate chains linked to the Fc region, wherein less than about 45%, about 40%, about 35%, about 30%, about 25%, about 20%, or about 15% of the N-glycoside-linked carbohydrate chains contain a fucose residue. In some aspects, provided herein is a formulation comprising an anti-Siglec-8 antibody described herein, wherein the antibody comprises a Fc region and N-glycoside-linked carbohydrate chains linked to the Fc region, wherein substantially none of the N-glycoside-linked carbohydrate chains contain a fucose residue. In some embodiments, one or two of the heavy chains of the antibody is non-fucosylated.

In one aspect, provided herein is a formulation (e.g., liquid formulation) suitable for an anti-Siglec-8 antibody comprising a heavy chain variable region and a light chain variable region, wherein the heavy chain variable region comprises (i) HVR-H1 comprising the amino acid sequence of SEQ ID NO:1, (ii) HVR-H2 comprising the amino acid sequence of SEQ ID NO:2, and (iii) HVR-H3 comprising the amino acid sequence of SEQ ID NO:3; and/or wherein the light chain variable region comprises (i) HVR-L1 comprising the amino acid sequence of SEQ ID NO:4, (ii) HVR-L2 comprising the amino acid sequence of SEQ ID NO:5, and (iii) HVR-L3 comprising the amino acid sequence of SEQ ID NO:6.

An anti-Siglec-8 antibody described herein may comprise any suitable framework variable domain sequence, provided that the antibody retains the ability to bind human Siglec-8. As used herein, heavy chain framework regions are designated “HC-FR1-FR4,” and light chain framework regions are designated “LC-FR1-FR4.” In some embodiments, the anti-Siglec-8 antibody comprises a heavy chain variable domain framework sequence of SEQ ID NOs:10, 11, 12, and 13 (HC-FR1, HC-FR2, HC-FR3, and HC-FR4, respectively). In some embodiments, the anti-Siglec-8 antibody comprises a light chain variable domain framework sequence of SEQ ID NOs:14, 15, 16, and 18 (LC-FR1, LC-FR2, LC-FR3, and LC-FR4, respectively). In some embodiments, the anti-Siglec-8 antibody comprises a light chain variable domain framework sequence of SEQ ID NOs:14, 15, 17, and 18 (LC-FR1, LC-FR2, LC-FR3, and LC-FR4, respectively).

There are five classes of immunoglobulins: IgA, IgD, IgE, IgG and IgM, having heavy chains designated α, δ, ε, γ and μ, respectively. The γ and α classes are further divided into subclasses e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1 or IgG4. In some embodiments, the antibody is an IgG1 antibody. In some embodiments, the antibody is an IgG4 antibody. In some embodiments, the human IgG4 comprises the amino acid substitution S228P, wherein the amino acid residues are numbered according to the EU index as in Kabat. In some embodiments, the human IgG1 comprises the amino acid sequence of SEQ ID NO:22. In some embodiments, the human IgG4 comprises the amino acid sequence of SEQ ID NO:23.

In some embodiments, provided herein is an anti-Siglec-8 antibody comprising a heavy chain comprising the amino acid sequence of SEQ ID NO:19; and/or a light chain comprising the amino acid sequence selected from SEQ ID NOs:20 or 21. In some embodiments, the antibody may comprise a heavy chain comprising the amino acid sequence of SEQ ID NO:27; and/or a light chain comprising the amino acid sequence of SEQ ID NO:20. In some embodiments, the anti-Siglec-8 antibody induces apoptosis of activated eosinophils. In some embodiments, the anti-Siglec-8 antibody induces apoptosis of resting eosinophils. In some embodiments, the anti-Siglec-8 antibody depletes activated eosinophils and inhibits mast cell activation. In some embodiments, the anti-Siglec-8 antibody depletes or reduces mast cells and inhibits mast cell activation. In some embodiments, the anti-Siglec-8 antibody depleted or reduces the number of mast cells. In some embodiments, the anti-Siglec-8 antibody kills mast cells by ADCC activity. In some embodiments, the antibody depletes or reduces mast cells expressing Siglec-8 in a tissue. In some embodiments, the antibody depletes or reduces mast cells expressing Siglec-8 in a biological fluid.

B. Excipients

Therapeutic formulations are prepared for storage by mixing the active ingredient (e.g., an anti-Siglec-8 antibody of the present disclosure) having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000). Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g., Zn-protein complexes); chelating agents such as EDTA and/or non-ionic surfactants.

In some embodiments, a formulation of the present disclosure comprises arginine. In some embodiments, the arginine is an arginine-HCl salt. In some embodiments, arginine is present in a formulation of the present disclosure in an amount or concentration of between about 50 mM and about 200 mM, about 100 mM to about 200 mM, or about 100 mM to about 150 mM. For example, in some embodiments, arginine can be present in a formulation of the present disclosure in an amount or concentration (in mM) that is greater than about 50, 60, 70, 80, 90, 100, 110, 120, 130, 140, 150, 160, 170, 180, or 190. In some embodiments, arginine can be present in a formulation of the present disclosure in an amount or concentration (in mM) that is less than about 200, 190, 180, 170, 160, 150, 140, 130, 120, 110, 100, 90, 80, 70, or 60. That is, a formulation of the present disclosure can comprise arginine in any amount or concentration (in mM) having a lower limit of about 50, 60, 70, 80, 90, 100, 110, 120, 130, 140, 150, 160, 170, 180, or 190 and an independently selected upper limit of about 200, 190, 180, 170, 160, 150, 140, 130, 120, 110, 100, 90, 80, 70, or 60, where the upper limit is greater than the lower limit. In some embodiments, a formulation of the present disclosure comprises arginine in an amount or concentration (in mM) of about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200.

In some embodiments, a formulation of the present disclosure comprises succinate. In some embodiments, the succinate is a sodium succinate salt. In some embodiments, the succinate is succinic acid (salt form). In some embodiments, succinate is present in a formulation of the present disclosure in an amount or concentration of between about 5 mM and about 50 mM, about 10 mM to about 30 mM, or about 10 mM to about 50 mM. For example, in some embodiments, succinate can be present in a formulation of the present disclosure in an amount or concentration (in mM) that is greater than about 5, 10, 15, 20, 25, 30, 35, 40, or 45. In some embodiments, succinate can be present in a formulation of the present disclosure in an amount or concentration (in mM) that is less than about 50, 45, 40, 35, 30, 25, 20, 15, or 10. That is, a formulation of the present disclosure can comprise succinate in any amount or concentration (in mM) having a lower limit of about 5, 10, 15, 20, 25, 30, 35, 40, or 45 and an independently selected upper limit of about 50, 45, 40, 35, 30, 25, 20, 15, or 10, where the upper limit is greater than the lower limit. In some embodiments, a formulation of the present disclosure comprises succinate in an amount or concentration (in mM) of about 5, about 10, about 15, about 20, about 25, about 30, about 35, about 40, about 45, or about 50.

In some embodiments, a formulation of the present disclosure comprises sodium chloride. In some embodiments, sodium chloride is present in a formulation of the present disclosure in an amount or concentration of between about 40 mM and about 150 mM, about 50 mM to about 130 mM, or about 75 mM to about 100 mM. For example, in some embodiments, sodium chloride can be present in a formulation of the present disclosure in an amount or concentration (in mM) that is greater than about 40, 50, 60, 70, 80, 90, 100, 110, 120, 130, or 140. In some embodiments, sodium chloride can be present in a formulation of the present disclosure in an amount or concentration (in mM) that is less than about 150, 140, 130, 120, 110, 100, 90, 80, 70, 60, or 50. That is, a formulation of the present disclosure can comprise sodium chloride in any amount or concentration (in mM) having a lower limit of about 40, 50, 60, 70, 80, 90, 100, 110, 120, 130, or 140 and an independently selected upper limit of about 150, 140, 130, 120, 110, 100, 90, 80, 70, 60, or 50, where the upper limit is greater than the lower limit. In some embodiments, a formulation of the present disclosure comprises sodium chloride in an amount or concentration (in mM) of about 40, about 45, about 50, about 55, about 60, about 65, about 70, about 75, about 80, about 85, about 90, about 95, about 100, about 105, about 110, about 115, about 120, about 125, about 130, about 135, about 140, about 145, or about 150.

In some embodiments, a formulation of the present disclosure comprises a non-ionic surfactant. Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody.

Suitable non-ionic surfactants include polysorbates (20, 40, 60, 65, 80, etc.) or polyoxyethylene sorbitan monoethers (TWEEN®-20, TWEEN®-80, etc.), polyoxamers (184, 188, etc.), PLURONIC® polyols, TRITON®, lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride. As used herein, references to a “polysorbate” can include polyoxyethylene sorbitan-based surfactants such as TWEEN®-20, TWEEN®-80, etc.

In some embodiments, a formulation of the present disclosure comprises polysorbate. In some embodiments, the polysorbate is polysorbate-20 or polysorbate-80. In some embodiments, polysorbate is present in a formulation of the present disclosure in an amount or concentration of between about 0.002% and about 0.05%, about 0.01% to about 0.05%, or about 0.02% to about 0.04% (w/v). For example, in some embodiments, polysorbate can be present in a formulation of the present disclosure in an amount or concentration (in %, w/v) that is greater than about 0.002, 0.005, 0.010, 0.015, 0.020, 0.025, 0.030, 0.035, 0.040, or 0.045. In some embodiments, polysorbate can be present in a formulation of the present disclosure in an amount or concentration (in %, w/v) that is less than about 0.05 or 0.050, 0.045, 0.040, 0.035, 0.030, 0.025, 0.020, 0.015, 0.010, or 0.005. That is, a formulation of the present disclosure can comprise polysorbate in any amount or concentration (in %, w/v) having a lower limit of about 0.002, 0.005, 0.010, 0.015, 0.020, 0.025, 0.030, 0.035, 0.040, or 0.045 and an independently selected upper limit of about 0.05 or 0.050, 0.045, 0.040, 0.035, 0.030, 0.025, 0.020, 0.015, 0.010, or 0.005, where the upper limit is greater than the lower limit. In some embodiments, a formulation of the present disclosure comprises polysorbate in an amount or concentration (in %, w/v) of about 0.002, about 0.005, about 0.010, about 0.015, about 0.020, about 0.025, about 0.030, about 0.035, about 0.040, about 0.045, or about 0.050.

In some embodiments, a formulation of the present disclosure has a pH of between about 5.0 and about 7.0, e.g., about 5.0, about 5.5, about 6.0, about 6.5, or about 7.0.

Buffers can be used to control the pH in a range which optimizes the therapeutic effectiveness (e.g., any of the ranges or pH values described supra), especially if stability is pH dependent. Buffers can be present at concentrations ranging from about 50 mM to about 250 mM. Suitable buffering agents for use with the present disclosure include both organic and inorganic acids and salts thereof, including those described supra. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Tonicity agents can be present in any amount between about 0.1% to about 25% by weight or between about 1 to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulations herein may also contain more than one active compound as necessary for the particular indication being treated, preferably those with complementary activities that do not adversely affect each other. Such active compounds are suitably present in combination in amounts that are effective for the purpose intended.

In some embodiments, a formulation of the present disclosure comprises: (a) an anti-Siglec-8 antibody of the present disclosure in an amount of between about 5 mg/mL and about 15 mg/mL, or between about 10 mg/mL and about 15 mg/mL; (b) arginine in an amount of between about 50 mM and about 200 mM, about 100 mM to about 200 mM, or about 100 mM to about 150 mM; (c) succinate in an amount of between about 5 mM and about 50 mM, about 10 mM to about 30 mM, or about 10 mM to about 50 mM; (d) sodium chloride in an amount of between about 40 mM and about 150 mM, about 50 mM to about 130 mM, or about 75 mM to about 100 mM; and (e) polysorbate in an amount of between about 0.002% and about 0.05%, about 0.01% to about 0.05%, or about 0.02% to about 0.04% (w/v), wherein the pH of the formulation is between about 5.0 and about 7.0. In some embodiments, a formulation of the present disclosure comprises: (a) an anti-Siglec-8 antibody of the present disclosure in an amount of between about 5 mg/mL and about 15 mg/mL; (b) arginine in an amount of between about 50 mM and about 200 mM; (c) succinate in an amount of between about 5 mM and about 50 mM; (d) sodium chloride in an amount of between about 40 mM and about 150 mM; and (e) polysorbate in an amount of between about 0.002% and about 0.05%, wherein optionally the pH of the formulation is between about 5.0 and about 7.0. In some embodiments, a formulation of the present disclosure comprises: (a) an anti-Siglec-8 antibody of the present disclosure in an amount of 15 mg/mL; (b) arginine in an amount of 125 mM; (c) succinate in an amount of 20 mM; (d) sodium chloride in an amount of 80 mM; and (e) polysorbate in an amount of 0.025%, wherein the pH of the formulation is 6.0. In some embodiments, the antibody comprises: (1) a heavy chain variable region comprising: an HVR-H1 comprising the amino acid sequence of SEQ ID NO:1; an HVR-H2 comprising the amino acid sequence of SEQ ID NO:2; an HVR-H3 comprising the amino acid sequence of SEQ ID NO:3; and (1) a light chain variable region comprising: an HVR-L1 comprising the amino acid sequence of SEQ ID NO:4; an HVR-L2 comprising the amino acid sequence of SEQ ID NO:5; and an HVR-L3 comprising the amino acid sequence of SEQ ID NO:6. In some embodiments, the antibody comprises a heavy chain comprising a heavy chain variable region comprising the amino acid sequence of SEQ ID NO:7, and a light chain comprising a light chain variable region comprising the amino acid sequence of SEQ ID NO:8 or SEQ ID NO:9. In some embodiments, the antibody comprises a heavy chain that comprises the amino acid sequence of SEQ ID NOs:19 or 27 and a light chain that comprises the amino acid sequence of SEQ ID NOs:20 or 21. In some embodiments, the formulation is a liquid formulation (e.g., liquid at a temperature of 2° C.-40° C.).

In some embodiments, a formulation of the present disclosure prevents or reduces antibody (e.g., an anti-Siglec-8 antibody of the present disclosure) aggregation and/or oligomerization. In some embodiments, percentage of aggregation refers to percentage of antibody present in a small antibody oligomer. In some embodiments, the abundance of small antibody oligomers is assayed by size-exclusion chromatography high-performance liquid chromatography. Without wishing to be bound to theory, it is thought that reduction in aggregation/oligomerization can improve formulation stability after freeze-thaw and/or agitation, which can occur during transportation or storage of therapeutic formulations, thereby maintaining the uniformity and/or efficacy of the formulation (e.g., the active component or drug of the formulation).

For example, in some embodiments, less than 5% of the antibody in the formulation is aggregated after freezing and thawing. Exemplary freeze-thaw conditions and assays for measuring antibody aggregation/oligomerization are described infra. In some embodiments, the formulation is subjected to freeze-thawing for one, two, three, four, or five cycles.

In some embodiments, less than 5% of the antibody in the formulation is aggregated after shaking or agitation. Exemplary agitation conditions and assays for measuring antibody aggregation/oligomerization are described infra. In some embodiments, the formulation is subjected to shaking overnight. In some embodiments, the formulation is subjected to shaking overnight at 800 rpm; for 2-4 days at 200 rpm; for 2-4 days at 500 rpm; or for 2 days at 200 rpm followed by 2 days at 500 rpm.

In some embodiments, the abundance of small antibody oligomers is assayed by UV-Vis spectroscopy. For example, in some embodiments, the absorbance at 400 nm (A400 nm) is measured. In some embodiments, the A400 nm of an antibody formulation is compared to that of a reference standard. In some embodiments, a reference standard refers to the corresponding formulation minus the antibody.

For example, in some embodiments, after freezing and thawing, a formulation of the present disclosure has an A400 nm of less than about 0.1, or an A400 nm of less than about 150% as compared to the A400 nm of a reference standard. Exemplary freeze-thaw conditions are described infra. In some embodiments, the formulation is subjected to freeze-thawing for one, two, three, four, or five cycles.

In some embodiments, after shaking or agitation, a formulation of the present disclosure has an A400 nm of less than about 0.1, or an A400 nm of less than about 150% as compared to the A400 nm of a reference standard. Exemplary agitation conditions are described infra. In some embodiments, the formulation is subjected to shaking overnight. In some embodiments, the formulation is subjected to shaking overnight at 800 rpm; for 2-4 days at 200 rpm; for 2-4 days at 500 rpm; or for 2 days at 200 rpm followed by 2 days at 500 rpm.

C. Administration

For the prevention or treatment of disease, the appropriate dosage of an active agent or formulation of the present disclosure, will depend on the type of disease to be treated, as defined above, the severity and course of the disease, whether the agent or formulation is administered for preventive or therapeutic purposes, previous therapy, the individual's clinical history and response to the agent, and the discretion of the attending physician. The agent or formulation is suitably administered to the individual at one time or over a series of treatments. In some embodiments, an interval between administrations of an anti-Siglec-8 antibody (e.g., an antibody that binds to human Siglec-8) or formulation described herein is about one month or longer. In some embodiments, the interval between administrations is about two months, about three months, about four months, about five months, about six months or longer. As used herein, an interval between administrations refers to the time period between one administration of the antibody and the next administration of the antibody. As used herein, an interval of about one month includes four weeks. Accordingly, in some embodiments, the interval between administrations is about four weeks, about five weeks, about six weeks, about seven weeks, about eight weeks, about nine weeks, about ten weeks, about eleven weeks, about twelve weeks, about sixteen weeks, about twenty weeks, about twenty four weeks, or longer. In some embodiments, the treatment includes multiple administrations of the antibody or formulation, wherein the interval between administrations may vary. For example, the interval between the first administration and the second administration is about one month, and the intervals between the subsequent administrations are about three months. In some embodiments, the interval between the first administration and the second administration is about one month, the interval between the second administration and the third administration is about two months, and the intervals between the subsequent administrations are about three months. In some embodiments, an anti-Siglec-8 antibody described herein (e.g., an antibody that binds to human Siglec-8) is administered at a flat dose. In some embodiments, an anti-Siglec-8 antibody described herein (e.g., an antibody that binds to human Siglec-8) is administered to an individual at a dosage from about 0.1 mg to about 1800 mg per dose. In some embodiments, the anti-Siglec-8 antibody (e.g., an antibody that binds to human Siglec-8) is administered to an individual at a dosage of about any of 0.1 mg, 0.5 mg, 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, and 1800 mg per dose. In some embodiments, an anti-Siglec-8 antibody described herein (e.g., an antibody that binds to human Siglec-8) is administered to an individual at a dosage from about 150 mg to about 450 mg per dose. In some embodiments, the anti-Siglec-8 antibody (e.g., an antibody that binds to human Siglec-8) is administered to an individual at a dosage of about any of 150 mg, 200 mg, 250 mg, 300 mg, 350 mg, 400 mg, and 450 mg per dose. In some embodiments, an anti-Siglec-8 antibody described herein (e.g., an antibody that binds to human Siglec-8) is administered to an individual at a dosage from about 0.1 mg/kg to about 20 mg/kg per dose. In some embodiments, an anti-Siglec-8 antibody described herein (e.g., an antibody that binds to human Siglec-8) is administered to an individual at a dosage from about 0.01 mg/kg to about 10 mg/kg per dose. In some embodiments, an anti-Siglec-8 antibody described herein (e.g., an antibody that binds to human Siglec-8) is administered to an individual at a dosage from about 0.1 mg/kg to about 10 mg/kg or about 1.0 mg/kg to about 10 mg/kg. In some embodiments, an anti-Siglec-8 antibody described herein is administered to an individual at a dosage of about any of 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg. Any of the dosing frequency described above may be used. Any dosing frequency described above may be used in the methods or uses of the compositions described herein. Efficacy of treatment with an antibody described herein (e.g., an antibody that binds to human Siglec-8) can be assessed using any of the methodologies or assays described herein at intervals ranging between every week and every three months. In some embodiments, efficacy of treatment (e.g., reduction or improvement of one or more symptoms) is assessed about every one month, about every two months, about every three months, about every four months, about every five months, about every six months or longer after administration of an antibody that binds to human Siglec-8. In some embodiments, efficacy of treatment (e.g., reduction or improvement of one or more symptoms) is assessed about every one week, about every two weeks, about every three weeks, about every four weeks, about every five weeks, about every six weeks, about every seven weeks, about every eight weeks, about every nine weeks, about every ten weeks, about every eleven weeks, about every twelve weeks, about every sixteen weeks, about every twenty weeks, about every twenty four weeks, or longer.

Antibodies described herein that bind to human Siglec-8 can be used either alone or in combination with other agents in the methods described herein. For instance, an antibody that binds to a human Siglec-8 may be co-administered with one or more (e.g., one or more, two or more, three or more, four or more, etc.) additional therapeutic agents for treating and/or preventing a Siglec-8-associated disease or disorder.

Such combination therapies noted above encompass combined administration (where two or more therapeutic agents are included in the same or separate formulations), and separate administration, in which case, administration of the antibody of the present disclosure can occur prior to, simultaneously, and/or following, administration of the one or more additional therapeutic agents. In some embodiments, administration of an anti-Siglec-8 antibody described herein and administration of one or more additional therapeutic agents occur within about one month, about two months, about three months, about four months, about five months or about six months of each other. In some embodiments, administration of an anti-Siglec-8 antibody described herein and administration of one or more additional therapeutic agents occur within about one week, about two weeks or about three weeks of each other. In some embodiments, administration of an anti-Siglec-8 antibody described herein and administration of one or more additional therapeutic agents occur within about one day, about two days, about three days, about four days, about five days, or about six days of each other.

Anti-Siglec8 antibodies and/or one or more additional therapeutic agents may be administered via any suitable route of administration known in the art, including, without limitation, by oral administration, sublingual administration, buccal administration, topical administration, rectal administration, via inhalation, transdermal administration, subcutaneous injection, intradermal injection, intravenous (IV) injection, intra-arterial injection, intramuscular injection, intracardiac injection, intraosseous injection, intraperitoneal injection, transmucosal administration, vaginal administration, intravitreal administration, intra-articular administration, peri-articular administration, local administration, epicutaneous administration, or any combinations thereof.

Biological Activity Assays

In some embodiments, an anti-Siglec-8 antibody described herein depletes eosinophils and inhibits mast cells. Assays for assessing apoptosis of cells are well known in the art, for example staining with Annexin V and the TUNNEL assay.

In some embodiments, an anti-Siglec-8 antibody described herein induces ADCC activity. In some embodiments, an anti-Siglec-8 antibody described herein kills eosinophils expressing Siglec-8 by ADCC activity. In some embodiments, a composition comprises non-fucosylated (i.e., afucosylated) anti-Siglec-8 antibodies. In some embodiments, a composition comprising non-fucosylated anti-Siglec-8 antibodies described herein enhances ADCC activity against Siglec-8 expressing eosinophils as compared to a composition comprising partially fucosylated anti-Siglec-8 antibodies. Assays for assessing ADCC activity are well known in the art and described herein. In an exemplary assay, to measure ADCC activity, effector cells and target cells are used. Examples of effector cells include natural killer (NK) cells, large granular lymphocytes (LGL), lymphokine-activated killer (LAK) cells and PBMC comprising NK and LGL, or leukocytes having Fc receptors on the cell surfaces, such as neutrophils, eosinophils and macrophages. Effector cells can be isolated from any source including individuals with a disease of interest (e.g., chronic urticaria). The target cell is any cell which expresses on the cell surface antigens that antibodies to be evaluated can recognize. An example of such a target cell is an eosinophil which expresses Siglec-8 on the cell surface. Another example of such a target cell is a cell line (e.g., Ramos cell line) which expresses Siglec-8 on the cell surface (e.g., Ramos 2C10)). Target cells can be labeled with a reagent that enables detection of cytolysis. Examples of reagents for labeling include a radio-active substance such as sodium chromate (Na₂ ⁵¹CrO₄). See, e.g., Immunology, 14, 181 (1968); J. Immunol. Methods., 172, 227 (1994); and J. Immunol. Methods., 184, 29 (1995).

In an exemplary assay to assess ADCC and apoptotic activity of anti-Siglec-8 antibodies on mast cells, human mast cells are isolated from human tissues or biological fluids according to published protocols (Guhl et al., Biosci. Biotechnol. Biochem., 2011, 75:382-384; Kulka et al., In Current Protocols in Immunology, 2001, (John Wiley & Sons, Inc.)) or differentiated from human hematopoietic stem cells, for example as described by Yokoi et al., J Allergy Clin Immunol., 2008, 121:499-505. Purified mast cells are resuspended in Complete RPMI medium in a sterile 96-well U-bottom plate and incubated in the presence or absence of anti-Siglec-8 antibodies for 30 minutes at concentrations ranging between 0.0001 ng/ml and 10 μg/ml. Samples are incubated for a further 4 to 48 hours with and without purified natural killer (NK) cells or fresh PBL to induce ADCC. Cell-killing by apoptosis or ADCC is analyzed by flow cytometry using fluorescent conjugated antibodies to detect mast cells (CD117 and Fc R1) and Annexin-V and 7AAD to discriminate live and dead or dying cells. Annexin-V and 7AAD staining are performed according to manufacturer's instructions.

In some aspects, an anti-Siglec-8 antibody described herein inhibits mast cell-mediated activities. Mast cell tryptase has been used as a biomarker for total mast cell number and activation. For example, total and active tryptase as well as histamine, N-methyl histamine, and 11-beta-prostaglandin F2 can be measured in blood or urine to assess the reduction in mast cells. See, e.g., U.S. Patent Application Publication No. US 20110293631 for an exemplary mast cell activity assay.

E. Antibody Preparation

The antibody described herein (e.g., an antibody that binds to human Siglec-8) is prepared using techniques available in the art for generating antibodies, exemplary methods of which are described in more detail in the following sections. Additional descriptions of techniques for generating antibodies can be found, e.g., in U.S. Pat. No. 9,546,215.

Antibody Fragments

The present disclosure encompasses antibody fragments. Antibody fragments may be generated by traditional means, such as enzymatic digestion, or by recombinant techniques. In certain circumstances there are advantages of using antibody fragments, rather than whole antibodies. For a review of certain antibody fragments, see Hudson et al. (2003) Nat. Med. 9:129-134.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. Fab and F(ab′)₂ fragment with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See WO 93/16185; U.S. Pat. Nos. 5,571,894; and 5,587,458. Fv and scFv are the only species with intact combining sites that are devoid of constant regions; thus, they may be suitable for reduced nonspecific binding during in vivo use. scFv fusion proteins may be constructed to yield fusion of an effector protein at either the amino or the carboxy terminus of an scFv. See Antibody Engineering, ed. Borrebaeck, supra. The antibody fragment may also be a “linear antibody”, e.g., as described in U.S. Pat. No. 5,641,870, for example. Such linear antibodies may be monospecific or bispecific.

Humanized Antibodies

The present disclosure encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent (e.g., mouse) antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those, skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human Antibodies

Human anti-Siglec-8 antibodies of the present disclosure can be constructed by combining Fv clone variable domain sequence(s) selected from human-derived phage display libraries with known human constant domain sequences(s). Alternatively, human monoclonal anti-Siglec-8 antibodies of the present disclosure can be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).

It is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255 (1993); Bruggermann et al., Year in Immunol., 7: 33 (1993).

Gene shuffling can also be used to derive human antibodies from non-human (e.g., rodent) antibodies, where the human antibody has similar affinities and specificities to the starting non-human antibody. According to this method, which is also called “epitope imprinting”, either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone, i.e., the epitope governs the choice of the human chain partner. When the process is repeated in order to replace the remaining non-human chain, a human antibody is obtained (see PCT WO 93/06213 published Apr. 1, 1993). Unlike traditional humanization of non-human antibodies by CDR grafting, this technique provides completely human antibodies, which have no FR or CDR residues of non-human origin.

Antibody Variants

In some embodiments, amino acid sequence modification(s) of the antibodies described herein are contemplated. For example, it may be desirable to improve the binding affinity and/or other biological properties of the antibody. Amino acid sequence variants of the antibody may be prepared by introducing appropriate changes into the nucleotide sequence encoding the antibody, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain. In some embodiments, truncated forms of monoclonal antibodies can be made by recombinant techniques.

In certain embodiments, an antibody of the present disclosure is altered to increase or decrease the extent to which the antibody is glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition or deletion of glycosylation sites to the antibody is conveniently accomplished by altering the amino acid sequence such that one or more of the above-described tripeptide sequences (for N-linked glycosylation sites) is created or removed. The alteration may also be made by the addition, deletion, or substitution of one or more serine or threonine residues to the sequence of the original antibody (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) on antigen-binding molecules with modified glycosylation.

In certain embodiments, a glycosylation variant comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87: 614 (2004). Examples of cell lines producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87: 614 (2004)), and cells overexpressing β1,4-N-acetylglycosminyltransferase III (GnT-III) and Golgi μ-mannosidase II (ManII).

Antibodies are contemplated herein that have reduced fucose relative to the amount of fucose on the same antibody produced in a wild-type CHO cell. For example, the antibody has a lower amount of fucose than it would otherwise have if produced by native CHO cells (e.g., a CHO cell that produce a native glycosylation pattern, such as, a CHO cell containing a native FUT8 gene). In certain embodiments, an anti-Siglec-8 antibody provided herein is one wherein less than about 50%, 40%, 30%, 20%, 10%, 5% or 1% of the N-linked glycans thereon comprise fucose. In certain embodiments, an anti-Siglec-8 antibody provided herein is one wherein none of the N-linked glycans thereon comprise fucose, i.e., wherein the antibody is completely without fucose, or has no fucose or is non-fucosylated or is afucosylated. The amount of fucose can be determined by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. In some embodiments, at least one or two of the heavy chains of the antibody is non-fucosylated.

In one embodiment, the antibody is altered to improve its serum half-life. To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule (US 2003/0190311, U.S. Pat. Nos. 6,821,505; 6,165,745; 5,624,821; 5,648,260; 6,165,745; 5,834,597).

Another type of variant is an amino acid substitution variant. These variants have at least one amino acid residue in the antibody molecule replaced by a different residue. Sites of interest for substitutional mutagenesis include the hypervariable regions, but FR alterations are also contemplated. Conservative substitutions are shown in Table 5 under the heading of “preferred substitutions.” If such substitutions result in a desirable change in biological activity, then more substantial changes, denominated “exemplary substitutions” in Table 5, or as further described below in reference to amino acid classes, may be introduced and the products screened.

TABLE 5

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or c) the bulk of the side chain. Amino acids may be grouped according to similarities in the properties of their side chains (in A. L. Lehninger, in Biochemistry, second ed., pp. 73-75, Worth Publishers, New York (1975)):
(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His (H)

Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites.

One type of substitutional variant involves substituting one or more hypervariable region residues of a parent antibody (e.g., a humanized or human antibody). Generally, the resulting variant(s) selected for further development will have modified (e.g., improved) biological properties relative to the parent antibody from which they are generated. A convenient way for generating such substitutional variants involves affinity maturation using phage display. Briefly, several hypervariable region sites (e.g., 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g., binding affinity). In order to identify candidate hypervariable region sites for modification, scanning mutagenesis (e.g., alanine scanning) can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to techniques known in the art, including those elaborated herein. Once such variants are generated, the panel of variants is subjected to screening using techniques known in the art, including those described herein, and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of antibodies of the present disclosure, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions including that of a hinge cysteine. In some embodiments, the Fc region variant comprises a human IgG4 Fc region. In a further embodiment, the human IgG4 Fc region comprises the amino acid substitution S228P, wherein the amino acid residues are numbered according to the EU index as in Kabat.

In accordance with this description and the teachings of the art, it is contemplated that in some embodiments, an antibody of the present disclosure may comprise one or more alterations as compared to the wild type counterpart antibody, e.g. in the Fc region. These antibodies would nonetheless retain substantially the same characteristics required for therapeutic utility as compared to their wild type counterpart. For example, it is thought that certain alterations can be made in the Fc region that would result in altered (i.e., either improved or diminished) C1q binding and/or Complement Dependent Cytotoxicity (CDC), e.g., as described in WO99/51642. See also Duncan & Winter Nature 322:738-40 (1988); U.S. Pat. Nos. 5,648,260; 5,624,821; and WO94/29351 concerning other examples of Fc region variants. WO00/42072 (Presta) and WO 2004/056312 (Lowman) describe antibody variants with improved or diminished binding to FcRs. The content of these patent publications are specifically incorporated herein by reference. See, also, Shields et al. J. Biol. Chem. 9(2): 6591-6604 (2001). Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). These antibodies comprise an Fc region with one or more substitutions therein which improve binding of the Fc region to FcRn. Polypeptide variants with altered Fc region amino acid sequences and increased or decreased C1q binding capability are described in U.S. Pat. No. 6,194,551B1, WO99/51642. The contents of those patent publications are specifically incorporated herein by reference. See, also, Idusogie et al. J. Immunol. 164: 4178-4184 (2000).

7. Vectors, Host Cells, and Recombinant Methods

For recombinant production of an antibody of the present disclosure, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

Generating Antibodies Using Eukaryotic Host Cells:

A vector for use in a eukaryotic host cell generally includes one or more of the following non-limiting components: a signal sequence, an origin of replication, one or more marker genes, an enhancer element, a promoter, and a transcription termination sequence.

a) Signal Sequence Component

A vector for use in a eukaryotic host cell may also contain a signal sequence or other polypeptide having a specific cleavage site at the N-terminus of the mature protein or polypeptide of interest. The heterologous signal sequence selected may be one that is recognized and processed (i.e., cleaved by a signal peptidase) by the host cell. In mammalian cell expression, mammalian signal sequences as well as viral secretory leaders, for example, the herpes simplex gD signal, are available. The DNA for such a precursor region is ligated in reading frame to DNA encoding the antibody.

b) Origin of Replication

Generally, an origin of replication component is not needed for mammalian expression vectors. For example, the SV40 origin may typically be used only because it contains the early promoter.

c) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, primate metallothionein genes, adenosine deaminase, ornithine decarboxylase, etc.

For example, in some embodiments, cells transformed with the DHFR selection gene are first identified by culturing all of the transformants in a culture medium that contains methotrexate (Mtx), a competitive antagonist of DHFR. In some embodiments, an appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199. Host cells may include NSO, CHOK1, CHOK1SV or derivatives, including cell lines deficient in glutamine synthetase (GS). Methods for the use of GS as a selectable marker for mammalian cells are described in U.S. Pat. Nos. 5,122,464 and 5,891,693.

d) Promoter Component

Expression and cloning vectors usually contain a promoter that is recognized by the host organism and is operably linked to nucleic acid encoding a polypeptide of interest (e.g., an antibody). Promoter sequences are known for eukaryotes. For example, virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. In certain embodiments, any or all of these sequences may be suitably inserted into eukaryotic expression vectors.

Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982), describing expression of human β-interferon cDNA in mouse cells under the control of a thymidine kinase promoter from herpes simplex virus. Alternatively, the Rous Sarcoma Virus long terminal repeat can be used as the promoter.

e) Enhancer Element Component

Transcription of DNA encoding an antibody of the present disclosure by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the human cytomegalovirus early promoter enhancer, the mouse cytomegalovirus early promoter enhancer, the polyoma enhancer on the late side of the replication origin, and adenovirus enhancers. See also Yaniv, Nature 297:17-18 (1982) describing enhancer elements for activation of eukaryotic promoters. The enhancer may be spliced into the vector at a position 5′ or 3′ to the antibody polypeptide-encoding sequence, but is generally located at a site 5′ from the promoter.

f) Transcription Termination Component

Expression vectors used in eukaryotic host cells may also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

g) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; CHOK1 cells, CHOK1SV cells or derivatives and a human hepatoma line (Hep G2).

Host cells are transformed with the above-described-expression or cloning vectors for antibody production and cultured in conventional nutrient media modified as appropriate for inducing promoters, selecting transformants, or amplifying the genes encoding the desired sequences.

h) Culturing the Host Cells

The host cells used to produce an antibody of the present disclosure may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other supplements may also be included at appropriate concentrations that would be known to those skilled in the art. The culture conditions, such as temperature, pH, and the like, are those previously used with the host cell selected for expression, and will be apparent to the ordinarily skilled artisan.

i) Purification of Antibody

When using recombinant techniques, the antibody can be produced intracellularly, or directly secreted into the medium. If the antibody is produced intracellularly, as a first step, the particulate debris, either host cells or lysed fragments, may be removed, for example, by centrifugation or ultrafiltration. Where the antibody is secreted into the medium, supernatants from such expression systems may be first concentrated using a commercially available protein concentration filter, for example, an Amicon or Millipore Pellicon ultrafiltration unit. A protease inhibitor such as PMSF may be included in any of the foregoing steps to inhibit proteolysis, and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a convenient technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Methods 62:1-13 (1983)). Protein G is recommended for all mouse isotypes and for human γ3 (Guss et al., EMBO J. 5:15671575 (1986)). The matrix to which the affinity ligand is attached may be agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to further purification, for example, by low pH hydrophobic interaction chromatography using an elution buffer at a pH between about 2.5-4.5, performed at low salt concentrations (e.g., from about 0-0.25M salt).

In general, various methodologies for preparing antibodies for use in research, testing, and clinical use are well-established in the art, consistent with the above-described methodologies and/or as deemed appropriate by one skilled in the art for a particular antibody of interest.

Production of Non-Fucosylated Antibodies

Provided herein are methods for preparing antibodies with a reduced degree of fucosylation. For example, methods contemplated herein include, but are not limited to, use of cell lines deficient in protein fucosylation (e.g., Lec13 CHO cells, alpha-1,6-fucosyltransferase gene knockout CHO cells, cells overexpressing β1,4-N-acetylglycosminyltransferase III and further overexpressing Golgi μ-mannosidase II, etc.), and addition of a fucose analog(s) in a cell culture medium used for the production of the antibodies. See Ripka et al. Arch. Biochem. Biophys. 249:533-545 (1986); US Pat Appl No US 2003/0157108 A1, Presta, L; WO 2004/056312 A1; Yamane-Ohnuki et al. Biotech. Bioeng. 87: 614 (2004); and U.S. Pat. No. 8,574,907. Additional techniques for reducing the fucose content of antibodies include Glymaxx technology described in U.S. Patent Application Publication No. 2012/0214975. Additional techniques for reducing the fucose content of antibodies also include the addition of one or more glycosidase inhibitors in a cell culture medium used for the production of the antibodies. Glycosidase inhibitors include α-glucosidase I, α-glucosidase II, and α-mannosidase I. In some embodiments, the glycosidase inhibitor is an inhibitor of α-mannosidase I (e.g., kifunensine).

As used herein, “core fucosylation” refers to addition of fucose (“fucosylation”) to N-acetylglucosamine (“GlcNAc”) at the reducing terminal of an N-linked glycan. Also provided are antibodies produced by such methods and compositions thereof.

In some embodiments, fucosylation of complex N-glycoside-linked sugar chains bound to the Fc region (or domain) is reduced. As used herein, a “complex N-glycoside-linked sugar chain” is typically bound to asparagine 297 (according to the number of Kabat), although a complex N-glycoside linked sugar chain can also be linked to other asparagine residues. A “complex N-glycoside-linked sugar chain” excludes a high mannose type of sugar chain, in which only mannose is incorporated at the non-reducing terminal of the core structure, but includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of the high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to the present methods, typically only a minor amount of fucose is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 1% of the antibody has core fucosylation by fucose in a composition. In some embodiments, substantially none (i.e., less than about 0.5%) of the antibody has core fucosylation by fucose in a composition. In some embodiments, more than about 40%, more than about 50%, more than about 60%, more than about 70%, more than about 80%, more than about 90%, more than about 91%, more than about 92%, more than about 93%, more than about 94%, more than about 95%, more than about 96%, more than about 97%, more than about 98%, or more than about 99% of the antibody is nonfucosylated in a composition.

In some embodiments, provided herein is an antibody wherein substantially none (i.e., less than about 0.5%) of the N-glycoside-linked carbohydrate chains contain a fucose residue. In some embodiments, provided herein is an antibody wherein at least one or two of the heavy chains of the antibody is non-fucosylated.

As described above, a variety of mammalian host-expression vector systems can be utilized to express an antibody. In some embodiments, the culture media is not supplemented with fucose. In some embodiments, an effective amount of a fucose analog is added to the culture media. In this context, an “effective amount” refers to an amount of the analog that is sufficient to decrease fucose incorporation into a complex N-glycoside-linked sugar chain of an antibody by at least about 10%, at least about 20%, at least about 30%, at least about 40% or at least about 50%. In some embodiments, antibodies produced by the instant methods comprise at least about 10%, at least about 20%, at least about 30%, at least about 40% or at least about 50% non-core fucosylated protein (e.g., lacking core fucosylation), as compared with antibodies produced from the host cells cultured in the absence of a fucose analog.

The content (e.g., the ratio) of sugar chains in which fucose is not bound to N-acetylglucosamine in the reducing end of the sugar chain versus sugar chains in which fucose is bound to N-acetylglucosamine in the reducing end of the sugar chain can be determined, for example, as described in the Examples. Other methods include hydrazinolysis or enzyme digestion (see, e.g., Biochemical Experimentation Methods 23: Method for Studying Glycoprotein Sugar Chain (Japan Scientific Societies Press), edited by Reiko Takahashi (1989)), fluorescence labeling or radioisotope labeling of the released sugar chain and then separating the labeled sugar chain by chromatography. Also, the compositions of the released sugar chains can be determined by analyzing the chains by the HPAEC-PAD method (see, e.g., J. Liq Chromatogr. 6:1557 (1983)). (See generally U.S. Patent Application Publication No. 2004/0110282.).

### III. Articles of Manufacture or Kits

In another aspect, an article of manufacture or kit is provided which comprises a formulation of the present disclosure comprising an anti-Siglec-8 antibody described herein (e.g., an antibody that binds human Siglec-8).

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic. The container holds the formulation. In some embodiments, the container is a glass vial. For example, in some embodiments, the glass vial contains 10 mg of the antibody, where the antibody is present in the formulation at a concentration of about 15 mg/mL.

The article of manufacture or kit may further comprise a label or a package insert, which is on or associated with the container, may indicate directions for use of the formulation. The label or package insert may further indicate that the formulation is useful or intended for subcutaneous, intravenous, or other modes of administration for treating and/or preventing a Siglec-8-associated disease or disorder in an individual. In some embodiments, the package insert comprises instructions for intravenous administration of the formulation. In some embodiments, the package insert comprises instructions for subcutaneous administration of the formulation. In some embodiments, the package insert further comprises instructions for the storing the anti-Siglec-8 antibody formulation, for example at between about 2° C. and about 8° C., e.g., at 4° C.

The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

Thus, in certain embodiments, the article of manufacture or kit comprises instructions for the use of an anti-Siglec-8 antibody formulation in methods for treating and/or preventing a Siglec-8-associated disease or disorder in an individual comprising administering to the individual an effective amount of the formulation. In certain embodiments, the article of manufacture comprises a medicament comprising an anti-Siglec-8 formulation and a package insert comprising instructions for administration of the medicament in an individual in need thereof to treat and/or prevent a Siglec-8-associated disease or disorder. In some embodiments, the package insert further indicates that the treatment is effective in reducing one or more symptoms in the individual with a Siglec-8-associated disease or disorder as compared to a baseline level before administration of the medicament. In some embodiments, the individual is diagnosed with a Siglec-8-associated disease or disorder before administration of the medicament comprising the antibody. In certain embodiments, the individual is a human.

In a specific embodiment, the present disclosure provides kits for a single dose-administration unit. Such kits comprise a container of an aqueous formulation of therapeutic antibody, including both single or multi-chambered pre-filled syringes. Exemplary pre-filled syringes are available from Vetter GmbH, Ravensburg, Germany.

In another embodiment, provided herein is an article of manufacture or kit comprising the formulations described herein for administration in an auto-injector device. An auto-injector can be described as an injection device that upon activation, will deliver its contents without additional necessary action from the patient or administrator. They are particularly suited for self-medication of therapeutic formulations when the delivery rate must be constant and the time of delivery is greater than a few moments.

In another aspect, an article of manufacture or kit is provided which comprises a formulation comprising an anti-Siglec-8 antibody described herein (e.g., an antibody that binds human Siglec-8). The article of manufacture or kit may further comprise instructions for use of the formulation in the methods of the present disclosure. Thus, in certain embodiments, the article of manufacture or kit comprises instructions for the use of a formulation comprising an anti-Siglec-8 antibody that binds to human Siglec-8 in methods for treating or preventing a Siglec-8-associated disease or disorder in an individual comprising administering to the individual an effective amount of the formulation. In certain embodiments, the article of manufacture or kit comprises a medicament comprising a formulation comprising an antibody that binds to human Siglec-8 and a package insert comprising instructions for administration of the medicament in an individual in need thereof to treat and/or prevent a Siglec-8-associated disease or disorder.

The present disclosure also provides an article of manufacture or kit which comprises a formulation comprising an anti-Siglec-8 antibody described herein (e.g., an antibody that binds human Siglec-8) in combination with one or more additional medicament (e.g., a second medicament) for treating or preventing a Siglec-8-associated disease or disorder in an individual. The article of manufacture or kit may further comprise instructions for use of the formulation in combination with one or more additional medicament in the methods of the present disclosure. For example, the article of manufacture or kit herein optionally further comprises a container comprising a second medicament, wherein the formulation comprising the anti-Siglec-8 antibody is a first medicament, and which article or kit further comprises instructions on the label or package insert for treating the individual with the second medicament, in an effective amount. Thus in certain embodiments, the article of manufacture or kit comprises instructions for the use of a formulation comprising an anti-Siglec-8 antibody that binds to human Siglec-8 in combination with one or more additional medicament in methods for treating or preventing a Siglec-8-associated disease or disorder in an individual. In certain embodiments, the article of manufacture or kit comprises a medicament comprising a formulation comprising an antibody that binds to human Siglec-8 (e.g., a first medicament), one or more additional medicament and a package insert comprising instructions for administration of the first medicament in combination with the one or more additional medicament (e.g., a second medicament).

It is understood that the aspects and embodiments described herein are for illustrative purposes only and that various modifications or changes in light thereof will be suggested to persons skilled in the art and are to be included within the spirit and purview of this application and scope of the appended claims.

### EXAMPLES

The present disclosure will be more fully understood by reference to the following examples. The examples should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: pH Screening for Anti-Siglec-8 Antibody Formulations

This Example describes a series of experiments that were undertaken to determine a pH at which anti-Siglec-8 formulations would exhibit highest stability without aggregation.

Materials and Methods

### pH Screening

Anti-Siglec-8 antibodies HEKA and HEKF were formulated at 10 mg/mL by dialyzing into each of the buffers shown in Table A. Liquid antibody formulations were subjected to 1 or 5 freeze-thaw cycles, or incubated at 4° C., 25° C., or 37° C. for 1 week, 2 weeks, 3 weeks, 1 month, 2 month, or 3 months, as indicated.

TABLE A
Formulations used for pH screening.

| | | Buffer | | | |
|---|---|---|---|---|---|
| Sample | IgG | Name | pH | Buffer | Salt |
| 1 | HEKA | pH 5 | 5 | 25 mM sodium Acetate | 150 mM NaCl |
| 2 | HEKA | pH 6 | 6 | 25 mM sodium Succinate | 150 mM NaCl |
| 3 | HEKA | pH 7 | 7 | 25 mM sodium Phosphate | 150 mM NaCl |
| 4 | HEKA | His (HBS) | 6 | 50 mM Histidine | 150 mM NaCl |
| 5 | HEKA | Arg (ABS) | 6 | 200 mM Arginine-HCl, 20 mM Succinate | 100 mM NaCl |
| 6 | HEKF | pH 5 | 5 | 25 mM sodium Acetate | 150 mM NaCl |
| 7 | HEKF | pH 6 | 6 | 25 mM sodium Succinate | 150 mM NaCl |
| 8 | HEKF | pH 7 | 7 | 25 mM sodium Phosphate | 150 mM NaCl |
| 9 | HEKF | His (HBS) | 6 | 50 mM Histidine | 150 mM NaCl |
| 10 | HEKF | Arg (ABS) | 6 | 200 mM Arginine-HCl, 20 mM Succinate | 100 mM NaCl |

### ELISA

An ELISA plate (Maxisorp 96-well 4004 flat-bottom clear plate) was coated with 100 μL of 0.1 μg/mL Siglec-8-ECD in 1×PBS, then incubated overnight at 4° C. The plate was washed four times with 300 μL 1×PBS+0.1% Tween-20, then blocked using 2% BSA in PBS-Tween at 200 μL/well while shaking at 600 rpm for 1 hour at room temperature. The plate was then washed again four times with 300 μL 1×PBS+0.1% Tween-20. A 5× dilution series of test antibody starting at 1.0 μg/mL (in blocking buffer) was then applied to the plate using 100 μL/well and incubated for 1 hour shaking at room temperature. The plate was then washed again four times with 300 μL 1×PBS+0.1% Tween-20. 100 μL secondary antibody (0.2 μg/mL goat anti-huFab:HRP in blocking buffer; Jackson Immunoresearch Catalog No. 115-035-071) was added to each well and incubated for 1 hour shaking at room temperature. The plate was then washed again four times with 300 μL 1×PBS+0.1% Tween-20. 100 μL TMB substrate (Sgima T0440-1L) was added to each well and developed for 5 minutes, then substrate development was stopped by adding 100 μL 1M sulfuric acid. ELISA plate was read at 450 nm.

### SEC-HPLC

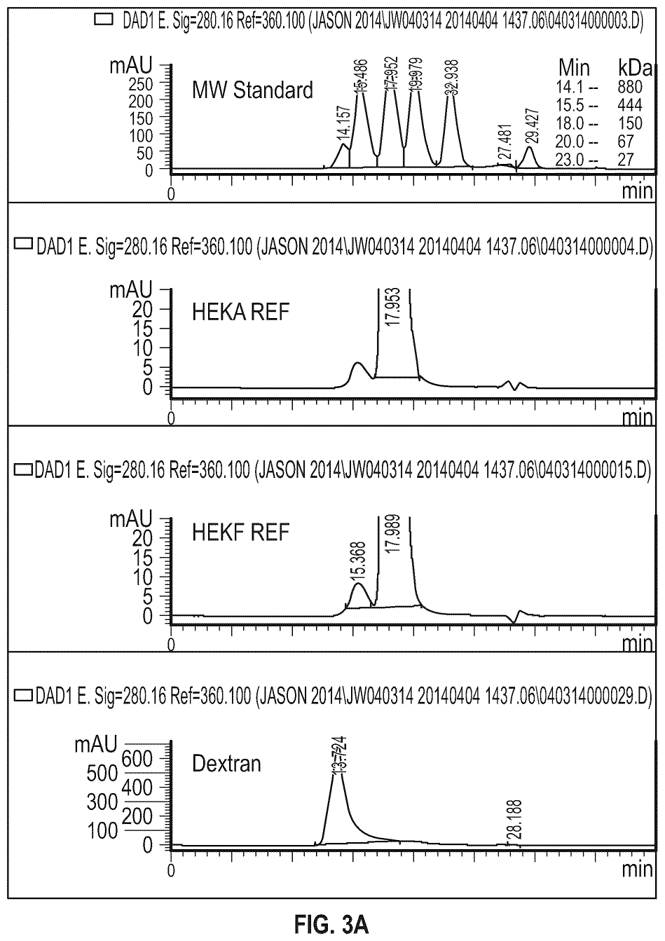

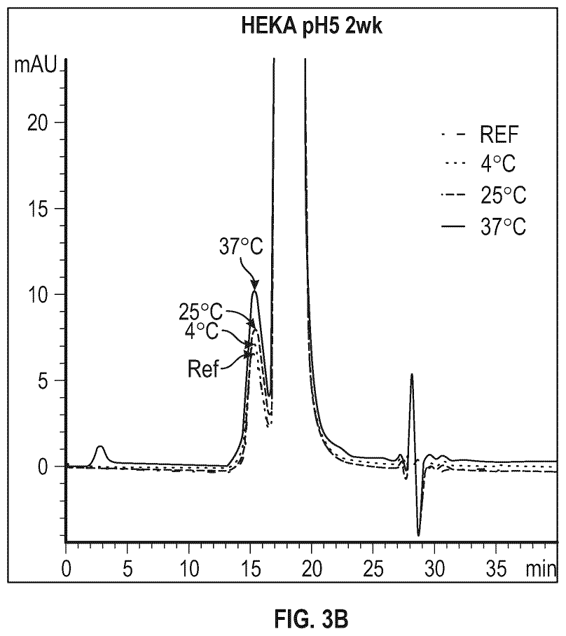

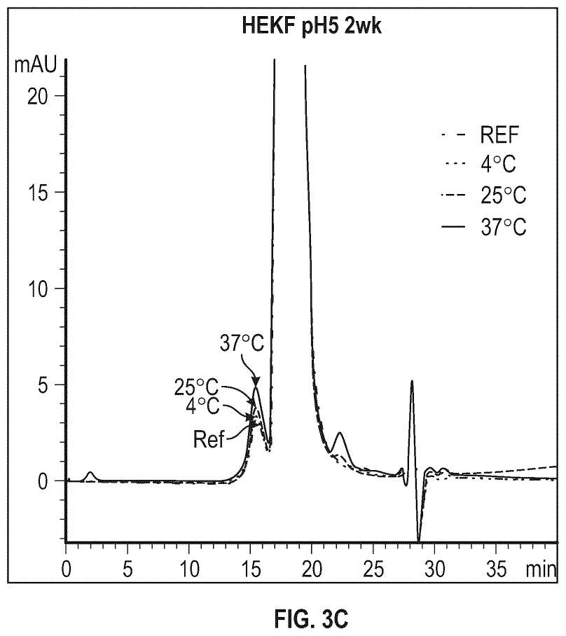

SUPERDEX™ 200 (GE Healthcare) 2.8/300 mm SEC columns were used at a flow rate of 0.075 mL/min in the indicated formulation buffer. Peaks were assigned as follows: 12.0 min—void; 13.5 min—dextran; 14.1 min—880 kD; 15.5 min—444 kD; 18.0 min—150 kD; 20.0 min—67 kD; 23.0 min—27 kD (FIG. 3A). Small anti-Siglec-8 antibody oligomers were observed in a peak at 15.4 min, whereas the antibody monomers were eluted at 18.0 min.

### DSC

Each respective formulation was used as a blank, and differential scanning calorimetry (DSC) was conducted at 60° C./hr., with monitoring from 20° C. to 110° C. (with 15 min. pre-scan). Formulations (see Table A) were incubated at 37° C. for 2 weeks, then analyzed.

### UV-Vis Spectroscopy

To measure presence of visible aggregates in the tested formulations, absorbance at 400 nm of solutions with antibodies was measured with a Perkin Elmer Lambda 35 UV-visible spectrometer. Absorbance measurements of the antibody solutions were subtracted from the formulation buffer.

### SDS-PAGE

Antibodies were analyzed by sodium dodecyl sulfate polyacrylamide gel electrophoresis (SDS-PAGE) on 4-20% Invitrogen gels, to assess stability and fragmentation. The analysis was performed under reducing and non-reducing conditions.

### Results

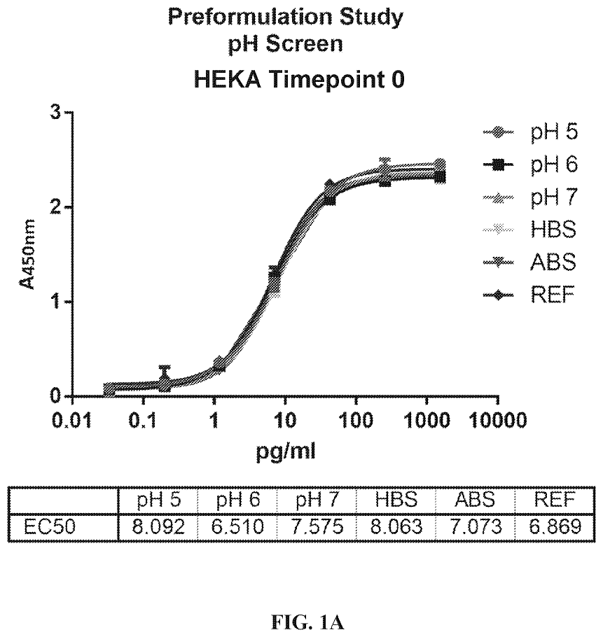

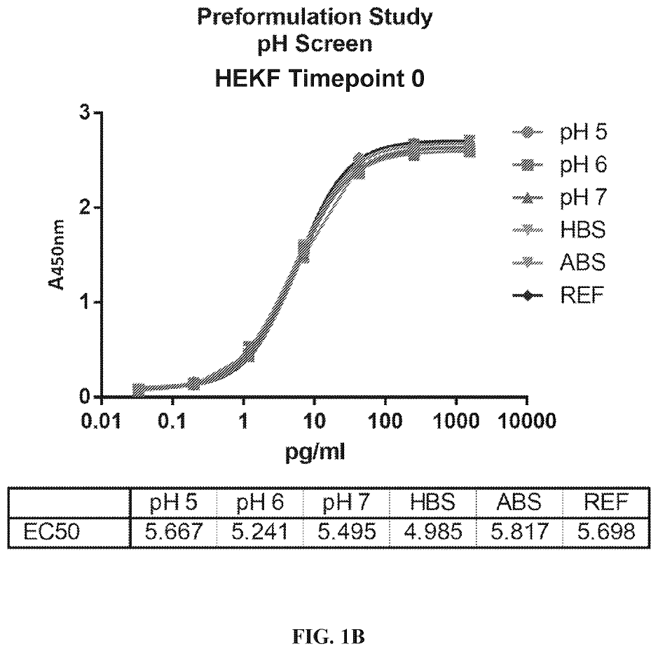

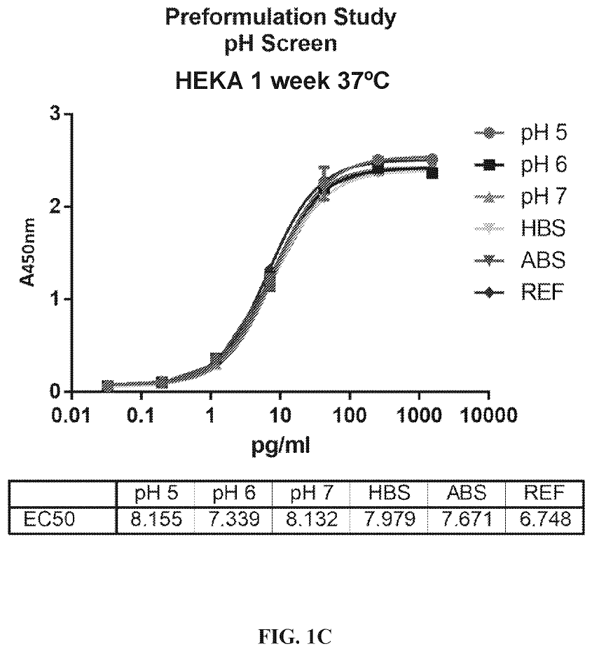

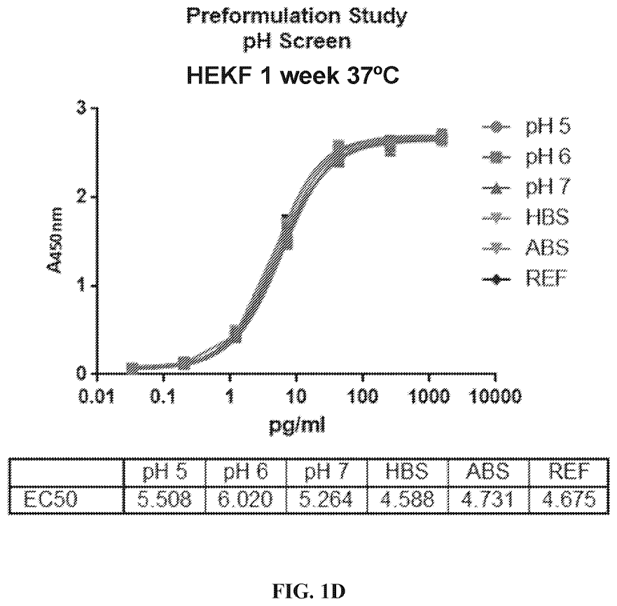

Anti-Siglec-8 antibodies were formulated as shown in Table A, subjected to freeze/thawing, and assayed for binding the Siglec-8-ECD by ELISA as described above. The binding curves and EC50 values for antibodies HEKA and HEKF at time 0 are shown in FIGS. 1A & 1B, respectively. Binding curves and EC50 values for the same antibody formulations after incubation for 1 week at 37° C. are shown in FIGS. 1C & 1D, respectively. Binding of the antibodies to Siglec-8 was not significantly affected by different pH value formulations.

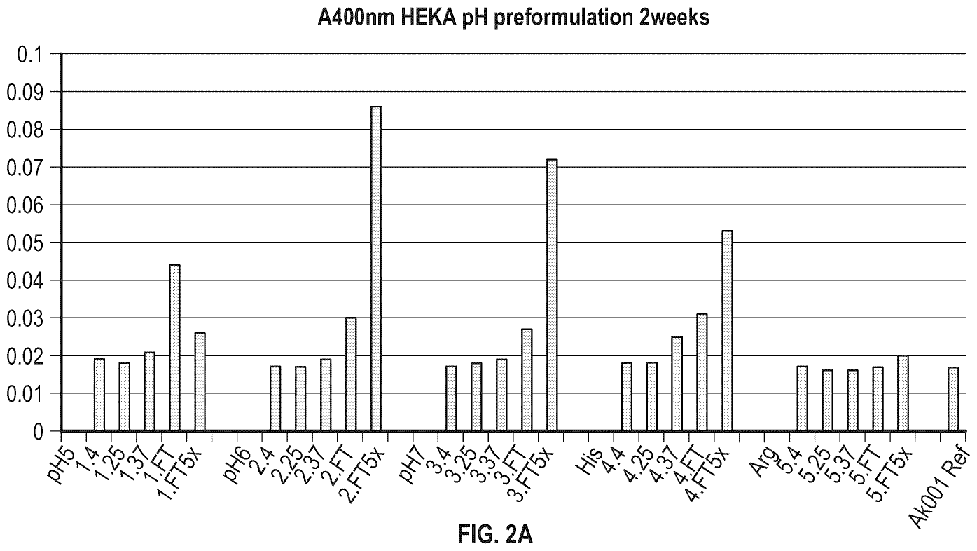

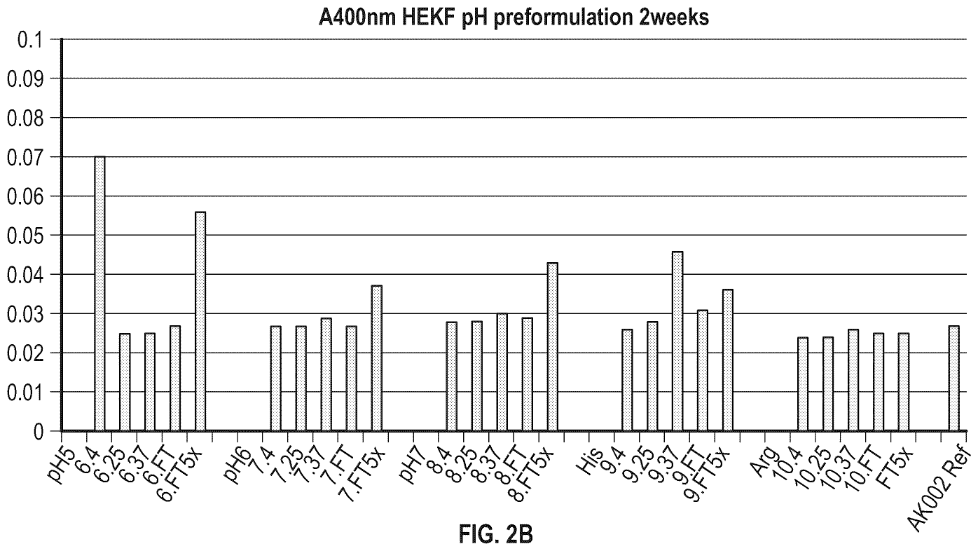

Next, UV-Vis spectroscopy was used to analyze the anti-Siglec-8 antibody formulations shown in Table A after storage at 2 weeks at the indicated temperature or freeze-thawing (1 or 5 cycles). Cuvettes were read at 1× at an absorbance of 400 nm. The results for HEKA and HEKF are shown in FIGS. 2A & 2B, respectively. Particles were visible after 5 freeze-thaw cycles in the pH 6, 7, and His HEKA formulations, but they did not settle and eventually dispersed. These data indicate that antibody HEKA was more stable at pH 6 in the presence of arginine.

SEC-HPLC was also used to quantify the formation of IgG monomers vs. small oligomers in anti-Siglec-8 antibody formulations. Some increase in antibody oligomers was observed at higher temperatures (FIGS. 3B & 3C), but at less than 1% of the total antibody. A smaller peak was observed for HEKF, which was also less than 1% (FIG. 3C). Shown are representative peaks obtained from pH 5 formulation after 2 weeks (see Table A). These data indicate that the presence of soluble aggregates was observed at pH 5 at elevated temperatures.

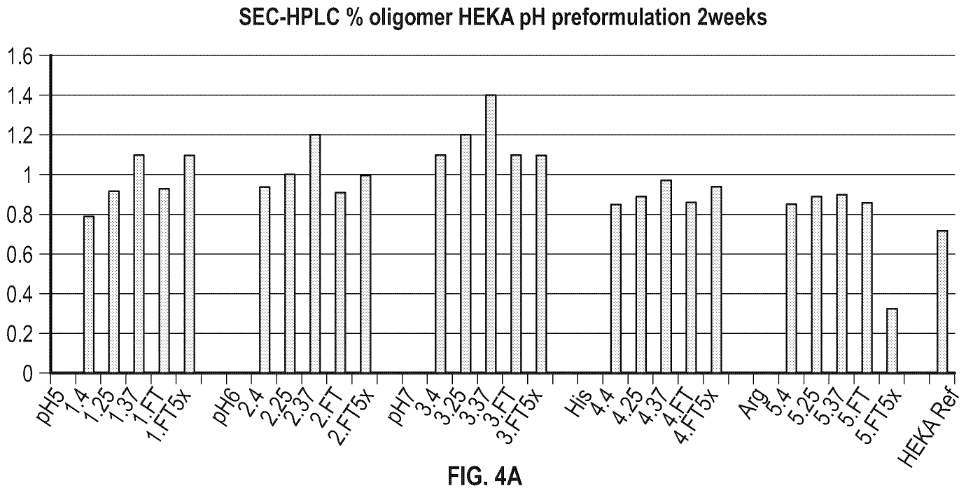

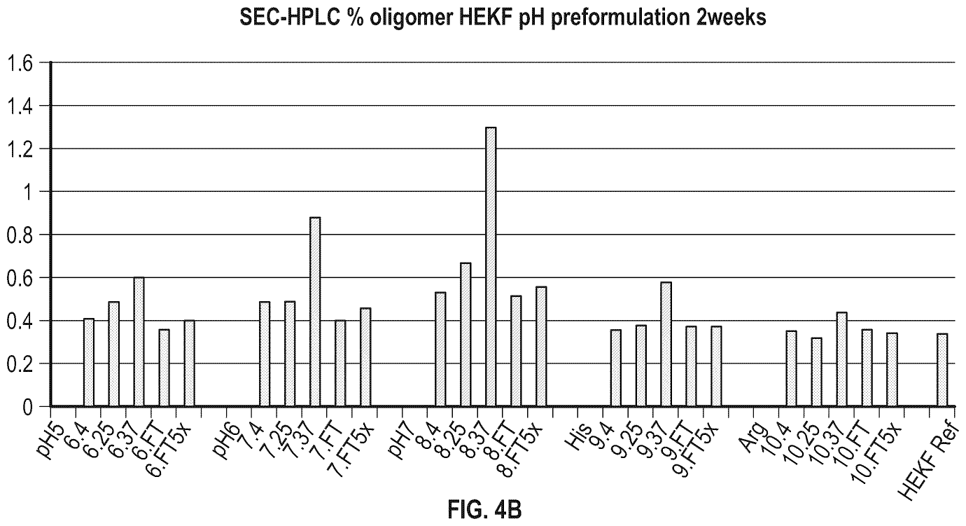

FIGS. 4A & 4B show the percentage of oligomers in each formulation with HEKA or HEKF, respectively. Formulations were tested after 2 weeks or after the indicated number of freeze-thaw cycles at the indicated temperature. These results confirmed the observation that some increase in antibody oligomers was seen at higher temperatures, but at less than 1% of the total antibody. These data suggest that arginine improves stability of the anti-Siglec-8 antibodies.

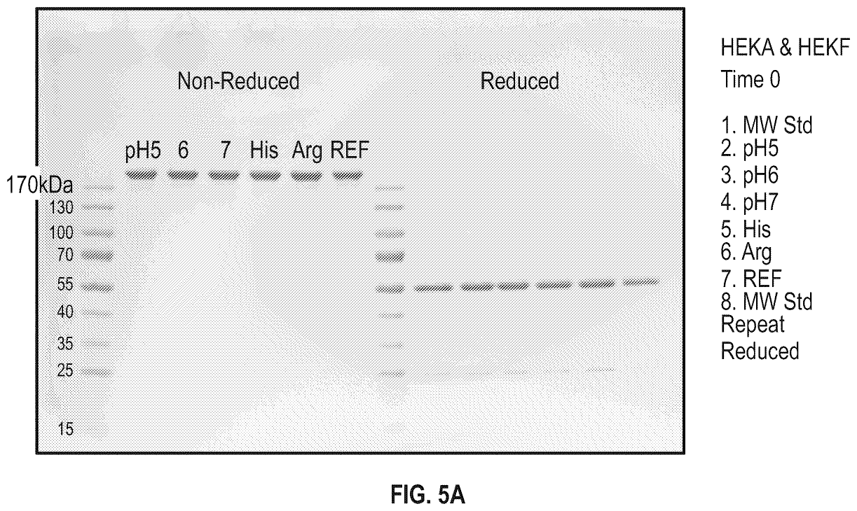

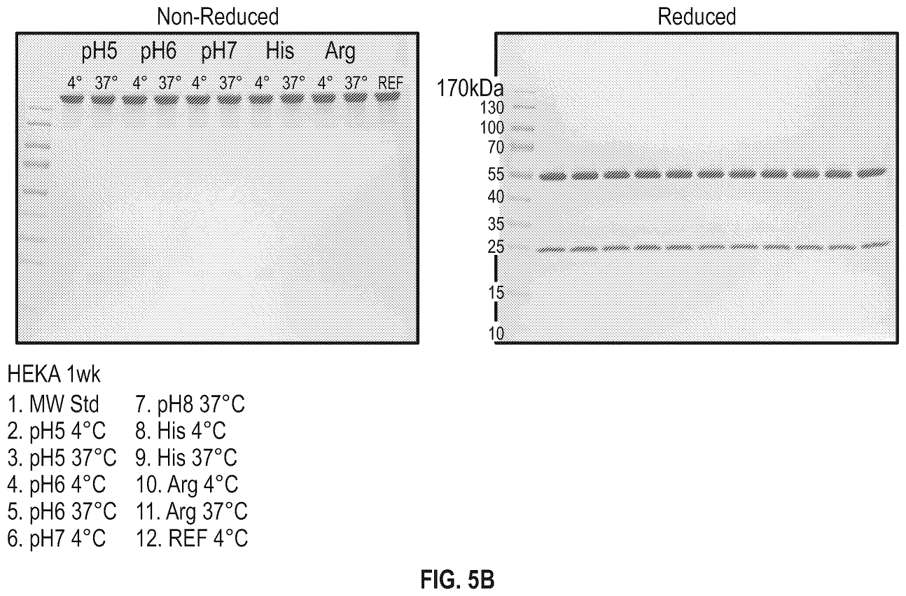

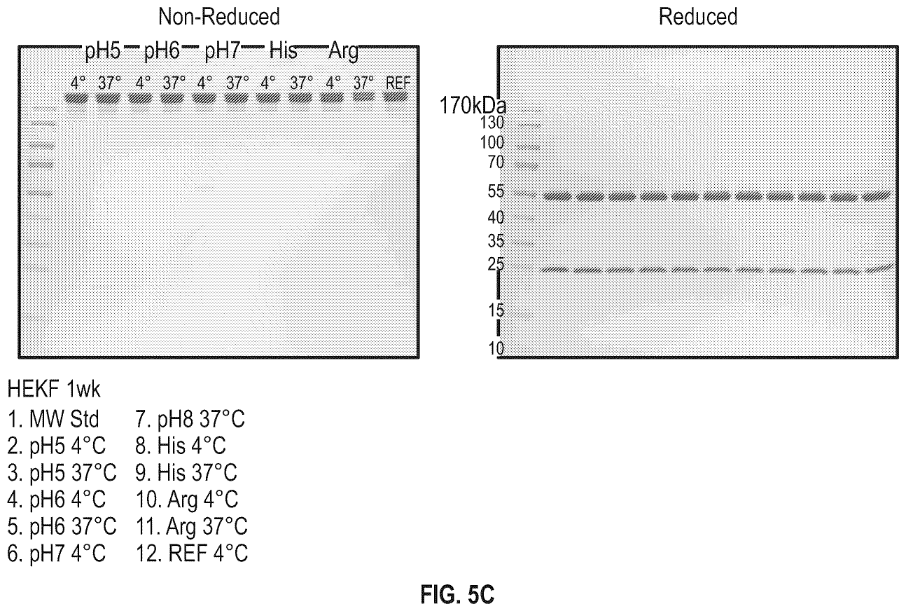

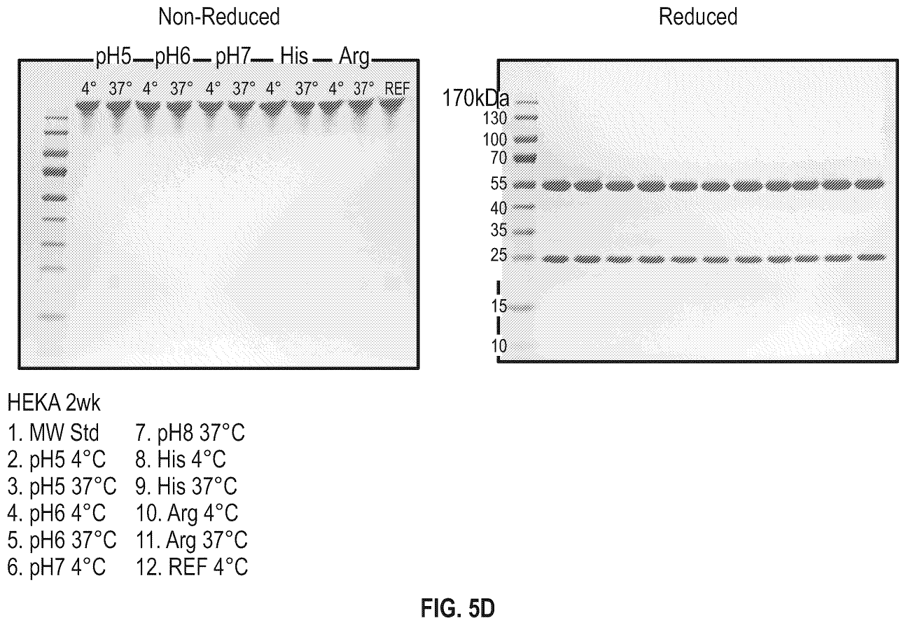

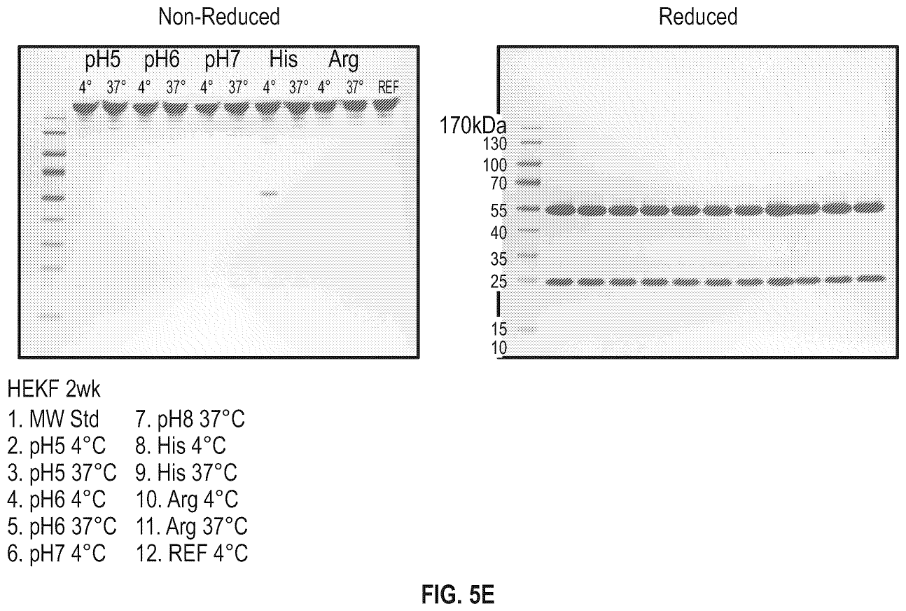

Anti-Siglec-8 antibody formulations were also examined by reducing and non-reducing SDS-PAGE. FIG. 5A shows the results of reducing and non-reducing SDS-PAGE analysis of the indicated HEKA and HEKF formulations at time 0. Analyses undertaken after 1 week or 2 weeks are shown in FIGS. 5B-5E. No significant fragmentation was observed under any of the conditions tested.

Differential scanning calorimetry (DSC) was also used to examine antibody stability. The results of DSC analysis and Tm peaks for HEKA and HEKF are shown in Tables B and C, respectively. The antibodies demonstrated better stability at pH 6 and in the presence of arginine.

TABLE B
DSC analysis of HEKA (IgG4) formulations.

| Buffer | Tm1 (° C.) | Tm2 (° C.) | Tm3 |
|---|---|---|---|
| REF | 66.6 | 72.3 | N/A |
| pH 6 | 68.1 | 73.1 | N/A |
| pH 7 | 69.3 | 73 | N/A |
| His pH 6 | 64.2 | 71.4 | N/A |
| Arg pH 6 | 66.6 | 72.2 | N/A |

TABLE C
DSC analysis of HEKF (IgG1) formulations.

| Buffer | Tm1 (° C.) | Tm2 (° C.) | Tm3 (° C.) |
|---|---|---|---|
| REF | 69.8 | 74.9 | 82.4 |
| pH 6 | 71.5 | 75.7 | 82.9 |
| pH 7 | 72.7 | 75.3 | 82.7 |
| His pH 6 | 67.4 | 74.9 | 81.7 |
| Arg pH 6 | 70.3 | 75.0 | 82.3 |

Taken together, these results demonstrate that the anti-Siglec-8 antibodies demonstrated better stability at pH 6, e.g., as compared to stability at pH 7.

### Example 2: Excipient Screening for Arginine Concentration in Anti-Siglec-8 Antibody Formulations

Various excipients were tested for their effect on antibody stability and aggregation in anti-Siglec-8 antibody formulations using the methods described in Example 1. First, a series of formulations was generated in order to test the effect of arginine concentration.

Results

The effect of arginine concentration on antibody aggregation was measured by UV-Vis spectroscopy. Anti-Siglec-8 antibodies were formulated according to the buffers shown in Table D at 10 mg/mL, then subjected to one or five freeze-thaw cycles and analyzed by UV-Vis spectroscopy.

TABLE D
Buffer formulations tested by UV-Vis spectroscopy.

| | Arginine | NaCl | Succinate | | |
|---|---|---|---|---|---|
| | Conc. | Conc. | Conc. | Conductivity | |
| Buffer | (mM) | (mM) | (mM) | (mS/cm) | pH |
| 1 | 0 | 130 | 20 | 20 | 6 |
| 2 | 50 | 120 | 20 | 20 | 6 |
| 3 | 100 | 75 | 20 | 20 | 6 |
| 4 | 150 | 50 | 20 | 20 | 6 |
| 5 | 200 | 0 | 20 | 20 | 6 |
| 6 | 100 | 40 | 20 | 16 | 6 |
| 7 | 100 | 75 | 20 (His) | 18 | 6 |
| 8 | 200 | 100 | 20 | 33 | 6 |

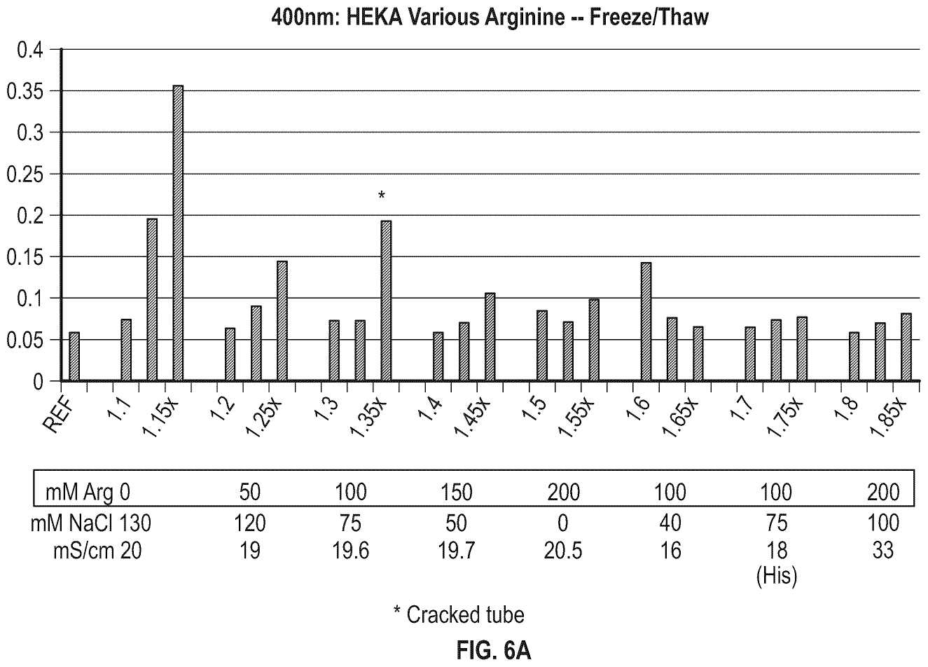

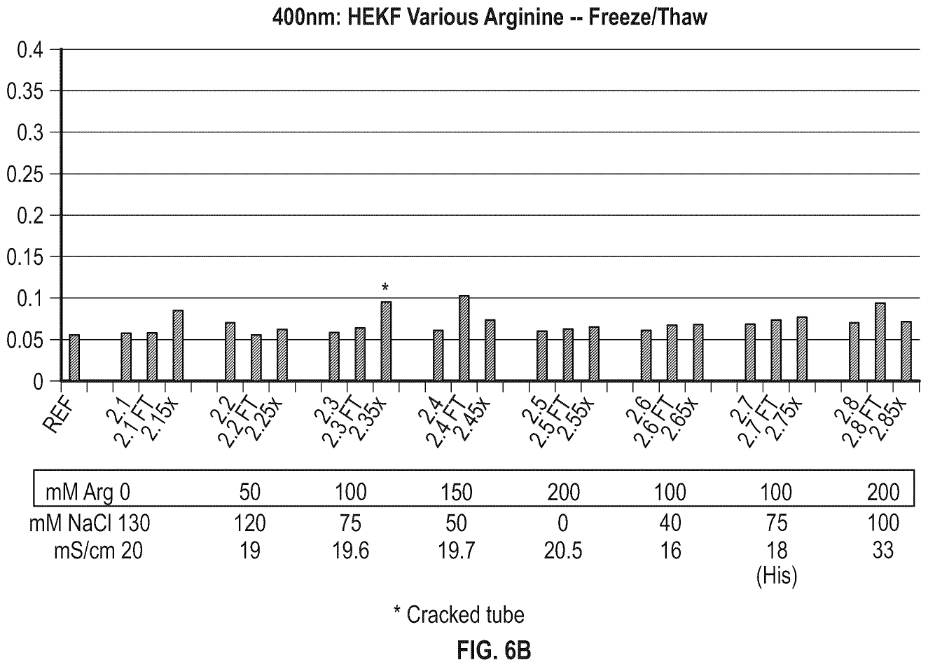

As shown in FIGS. 6A & 6B, the presence of 50-200 mM Arginine in anti-Siglec-8 antibody formulations reduced aggregation of the HEKA and HEKF antibodies after freeze-thaw.

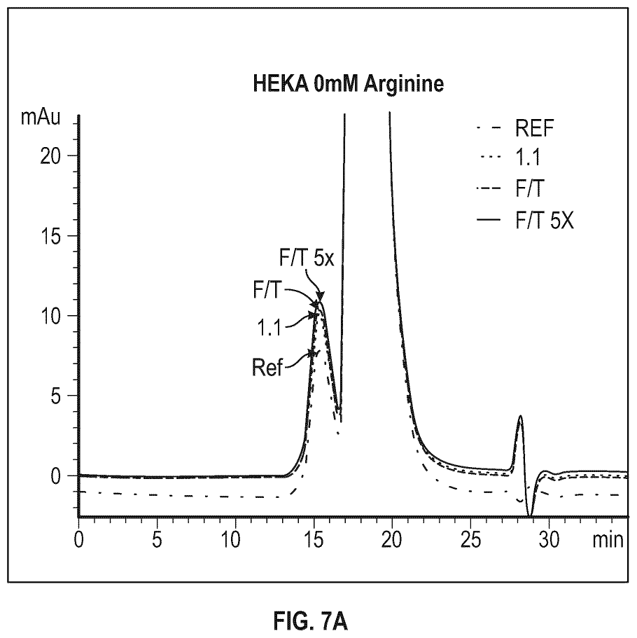

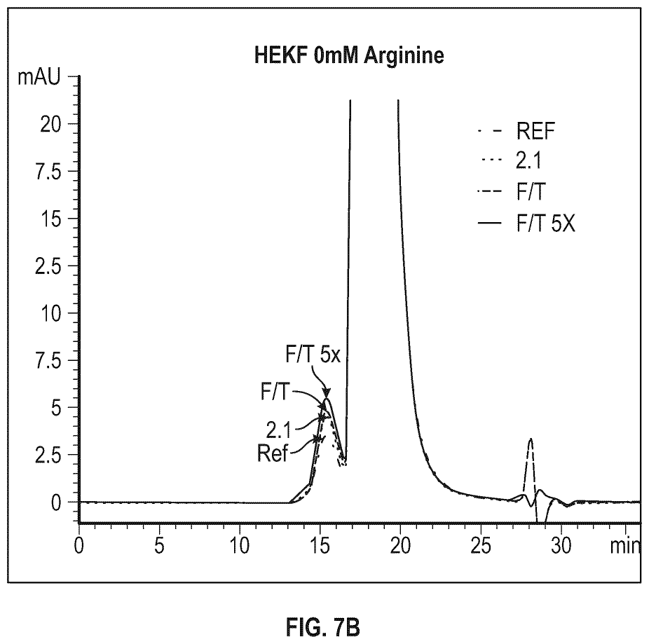

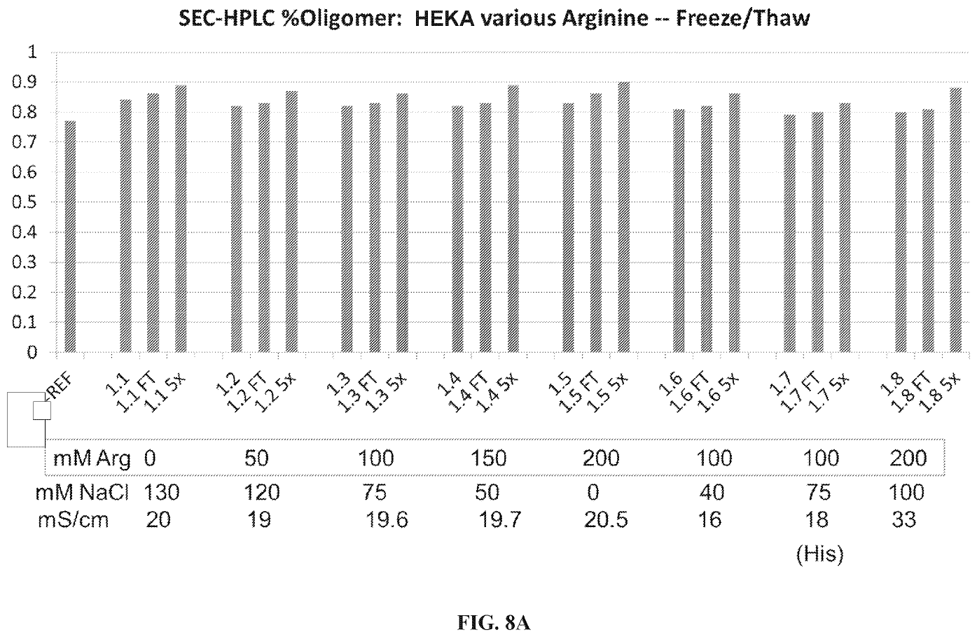

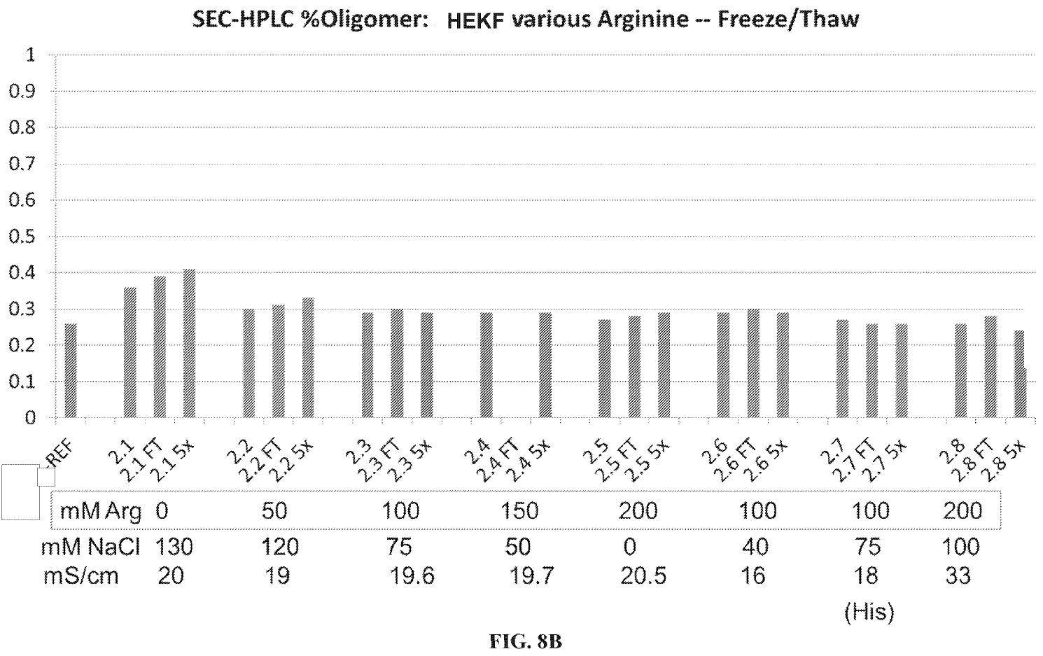

Antibody oligomerization of the formulations shown in Table D was also analyzed by SEC-HPLC as described above. Representative peaks for formulation 1 are shown in FIGS. 7A & 7B. These results demonstrate a very small increase (≤0.1%) of small antibody oligomers after freeze-thawing for both anti-Siglec-8 antibodies. The results for all formulations tested are shown in FIGS. 8A (antibody HEKA) and 8B (antibody HEKF). The effects of arginine at concentrations above 100 mM were clear and sustained at 150 mM.

Taken together, these results show that the presence of arginine in anti-Siglec-8 antibody formulations, and in particular concentrations of arginine over 100 mM, reduce antibody aggregation and oligomerization.

### Example 3: Agitation Study for Sucrose, Succinate, and Polysorbate Concentrations in Anti-Siglec-8 Antibody Formulations

Various excipients were tested for their effect on antibody stability and aggregation in anti-Siglec-8 antibody formulations. Next, a series of formulations was generated in order to test the effect of sucrose, succinate, and polysorbate concentrations. Sucrose and polysorbate were used to help stability in these agitation studies.

Results

A series of buffers was constructed to test the effect of polysorbate concentration on antibody performance after agitation. These buffers included 100 mM arginine, 40 mM NaCl, 20 mM succinate, and one of the following concentrations of polysorbate-80: 0%, 0.002%, 0.005%, 0.01%, 0.02%, and 0.05%. HEKA antibody was formulated at 10 mg/mL, and liquid formulations were aliquotted into sterile glass formulation vials. Vials were shaken horizontally for 2 days at 200 rpm, then accelerated to 500 rpm for 2 additional days. After 4 days, absorbance at 400 nm was assayed.

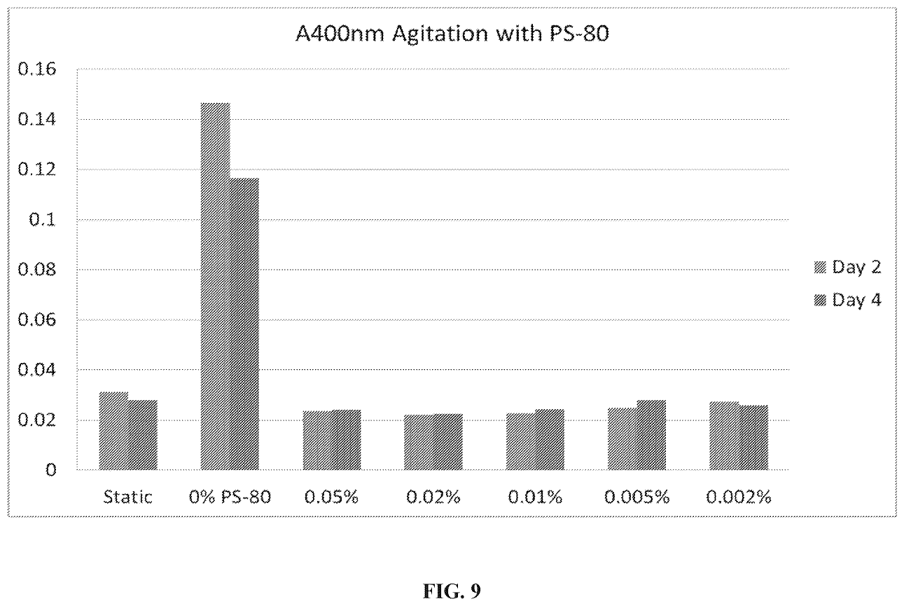

The results of the agitation experiments are shown in FIG. 9. These results demonstrated a low absorbance value for all HEKA formulations containing polysorbate.

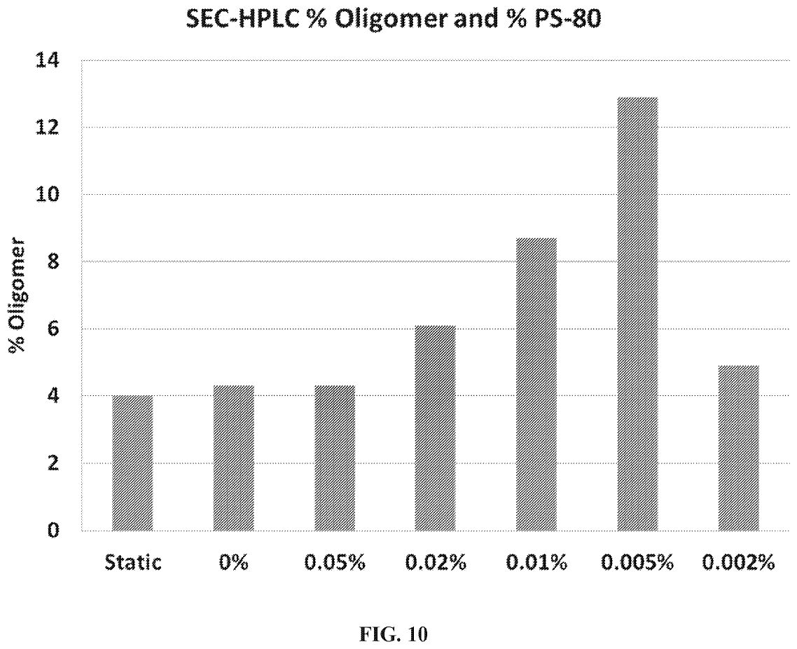

Polysorbate-containing formulations were next analyzed by SEC-HPLC for the formation of small antibody oligomers. The results are summarized in FIG. 10 and Table E.

TABLE E
Oligomer formation as a function of formulation
polysorbate concentration.

| Sample | % oligomer | oligomer | monomer | total | % REF |
|---|---|---|---|---|---|
| Static | 4 | 2570 | 59150 | 61720 | 100.0 |
| 0% PS80 | 4.3 | 2612 | 57930 | 60542 | 98.1 |
| 0.05% | 4.3 | 2613 | 58750 | 61363 | 99.4 |
| 0.02% | 6.1 | 3714 | 57430 | 61144 | 99.1 |
| 0.01% | 8.7 | 5454 | 57022 | 62476 | 101.2 |
| 0.05% | 12.9 | 8049 | 54167 | 62216 | 100.8 |
| 0.002% | 4.9 | 3054 | 59100 | 62154 | 100.7 |

While the absorbance values at 400 nm did not vary significantly with polysorbate concentration (FIG. 9), the presence of oligomerization was found to be sensitive to polysorbate levels (FIG. 10 and Table E). The proportion of antibody oligomers increased with decreasing concentrations of polysorbate until 0.005%, but lower oligomer levels were observed at 0.002% and 0% polysorbate.

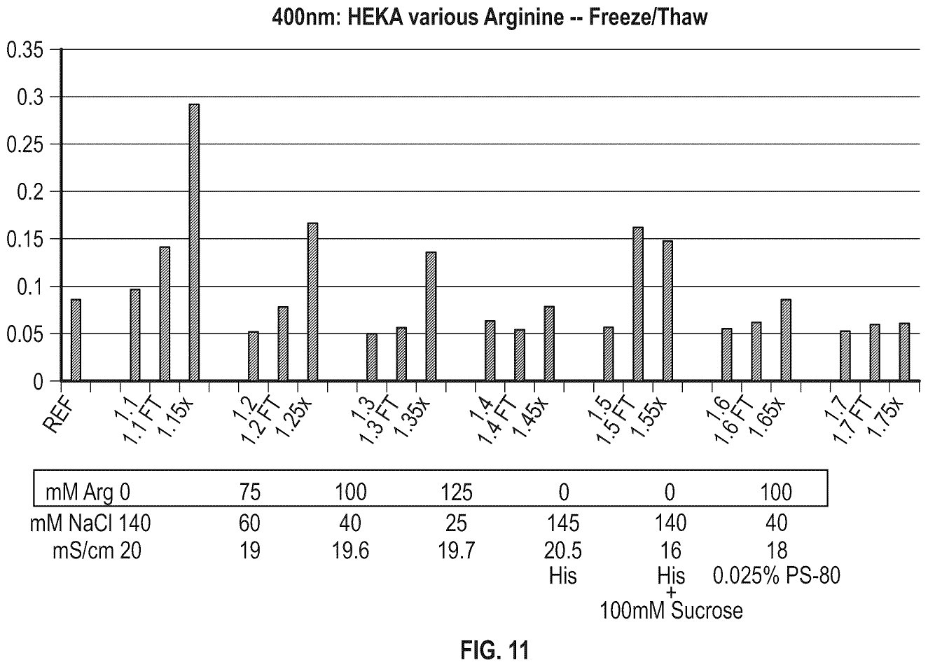

Next, a series of formulations was generated to test the effects of arginine, histidine, sucrose, and polysorbate on antibody aggregation, as measured by A400 nm. Histidine was used at 50 mM. The HEKA formulations tested and results obtained are shown in FIG. 11. These results demonstrated that formulations with arginine or histidine resulted in less aggregation, and that sucrose and polysorbate improved antibody stability upon freezing.

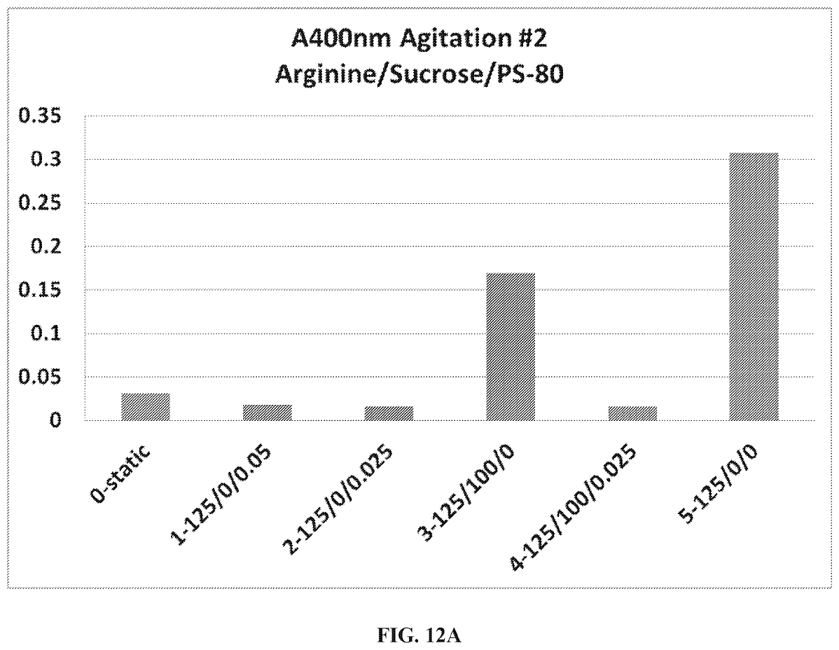

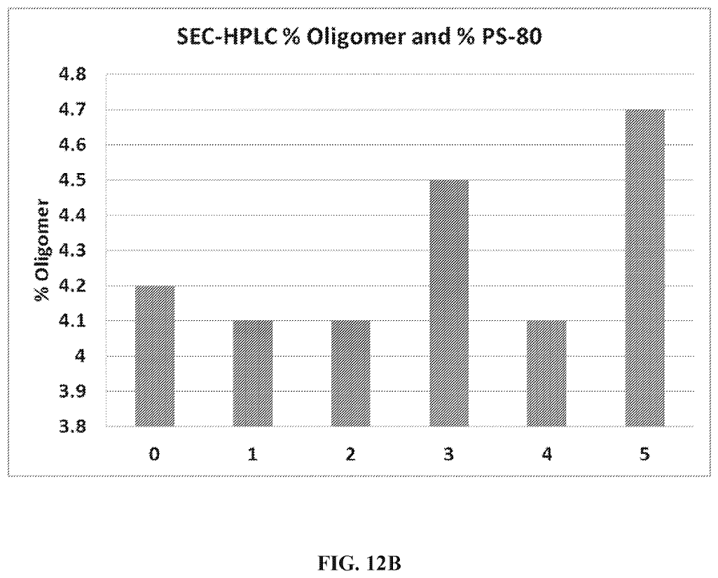

Next, the effect of arginine, sucrose, and polysorbate concentrations were examined after HEKA antibody agitation. Agitation was conducted as described above, except that vials were agitated at 800 rpm for 1 day. Formulations tested, as well as A400 nm values, are shown in Table F. The results are shown in FIG. 12A and Table F.

TABLE F
Formulations tested in agitation experiments.

| Vial | mM Arginine | mM Sucrose | % PS-80 | A400 |
|---|---|---|---|---|
| 0 | 0 | 0 | 0 | 0.0309 |
| 1 | 125 | 0 | 0.05 | 0.0179 |
| 2 | 125 | 0 | 0.025 | 0.0165 |
| 3 | 125 | 100 | 0 | 0.1695 |
| 4 | 125 | 100 | 0.025 | 0.0161 |
| 5 | 125 | 0 | 0 | 0.3076 |

These results demonstrated that the presence of polysorbate in antibody formulations reduced HEKA aggregation.

These formulations were also analyzed for the formation of small antibody oligomers by SEC-HPLC (FIG. 12B). Vials with no polysorbate-80 showed turbidity and larger oligomers after agitation. Using 100 mM sucrose without polysorbate-80 also resulted in turbidity. These results demonstrated that the presence of arginine and polysorbate resulted in antibody formulations with less oligomerization after agitation.

HEKA formulations were also tested for aggregation and oligomerization after freeze-thaw. Formulations were generated and subjected to 1 freeze-thaw cycle (F/T), 5 freeze-thaw cycles (F/T 5×), or agitation (Agit) according to Table G. These formulations included 125 mM Arginine-HCl, 25 mM NaCl, and 20 mM sodium succinate, pH6.

TABLE G
Formulations tested in agitation and freeze-thaw experiments.

| Vial | % PS-80 | Sample | A400 | % oligo |
|---|---|---|---|---|
| 0 | 0 | Static | 0.0234 | 4.3 |
| 1 | 0.01 | F/T | 0.0274 | 4.3 |
| 1 | 0.01 | F/T 5X | 0.0269 | 4.3 |
| 1 | 0.01 | Agit | 0.0313 | 4.8 |
| 2 | 0.02 | F/T | 0.0251 | 4.3 |
| 2 | 0.02 | F/T 5X | 0.0262 | 4.3 |
| 2 | 0.02 | Agit | 0.0252 | 4.3 |
| 3 | 0.03 | F/T | 0.0239 | 4.3 |
| 3 | 0.03 | F/T 5X | 0.0243 | 4.3 |
| 3 | 0.03 | Agit | 0.0239 | 4.3 |

As shown in Table G, the absorbance values and percentages of oligomerization were consistently low in all formulations, with a slight increase in oligomerization observed for the lowest concentration of polysorbate-80 after agitation.

An additive effect of sucrose and polysorbate with arginine with observed at pH 6. The agitation stability data indicate that the formulation prevented aggregation of the antibody if vials are agitated (e.g., during transportation) and/or if the material is freeze-thawed. Without wishing to be bound to theory, it is thought that sucrose behaves as a cryo-protectant, arginine behaves as an antibody stabilizer, and polysorbate acts to minimize aggregation.

### Example 4: Component Exclusion and Target Weight Studies on Anti-Siglec-8 Antibody Formulations

The goal of this Example was to confirm the function and impact of each component in an anti-Siglec-8 formulation buffer comprising 125 mM arginine, 80 mM sodium Chloride, 20 mM succinate, and 0.025% polysorbate 80, pH 6.0.

Formulations were prepared with all of the above components as a control, or formulations with each of the components excluded, one at a time (1 L in duplicates). Formulation buffers were prepared at low end (20% less) and high end (20% more) of target weight of all components. pH and conductivity were measured at 22.0-24.0° C.

The results are shown in Table H. Polysorbate 80 was not evaluated in this study since the addition volumes are minute and do not have an effect on the parameters studied.

TABLE H
Component exclusion study results.

| Excluded | Control (none | L- | L-arginine | Sodium | Succinic |
|---|---|---|---|---|---|
| component | excluded) | arginine | HCl | chloride | acid |
| pH | 5.93 | 3.32 | 6.04 | 6.06 | 8.76 |
| Conductivity | 15.70 | 14.57 | 9.90 | 8.39 | 14.29 |
| (mS/cm) | | | | | |

The results demonstrated that the exclusion of L-arginine led to a decrease in pH. Exclusion of L-arginine HCl or sodium chloride led to a decrease in conductivity. Exclusion of succinic acid led to an increase in pH.

Without wishing to be bound to theory, it is thought that in these formulations, L-arginine and L-arginine HCl act as a buffer/stabilizer, succinic acid acts as a buffer, sodium chloride provides isotonicity, polysorbate 80 acts as a surfactant, and water for injection is the solvent.

Studies were also undertaken to examine the effect of ±20% target weight variation on the buffer preparation. The results are shown in Table I.

TABLE I
±20% target weight buffer preparation results

| Target Weight % | *Control | 20% less | 20% more |
|---|---|---|---|
| pH | 5.93 | 6.02 | 5.94 |
| Conductivity | 15.7 | 12.74 | 17.99 |
| (mS/cm) | | | |

The results showed no impact on pH due to changes in the proportion of components. However, a direct impact to conductivity was observed at each end of the range. Conductivity specification range was 13.35-16.35 mS/cm at 22.0-24.0° C., and pH specification range was 5.90-6.10.

These studies confirmed the function of each component of this formulation buffer. Exclusion of any of the components would result in the buffer being out of spec for pH or conductivity. The target weight experiments indicated that conductivity would fall out of specification on either end.

### Example 5: Freeze/Thaw and Agitation Studies for Polysorbate Concentration in Anti-Siglec-8 Antibody Formulations

The goals of this Example were to evaluate the freeze/thaw and agitation impacts on four anti-Siglec-8 antibody formulations prepared with polysorbate 80 concentrations of 0.0%, 0.015%, 0.025%, and 0.030%.

5 freeze/thaw cycles were carried out using HDPE bottles (12 samples total) with the following formulations:
Anti-Siglec-8 HEKA at 15 mg/mL with 0.0% PS80 at 1×, 3×, and 5× freeze/thaw cycles
Anti-Siglec-8 HEKA at 15 mg/mL with 0.015% PS80 at 1×, 3×, and 5× freeze/thaw cycles
Anti-Siglec-8 HEKA at 15 mg/mL with 0.025% PS80 at 1×, 3×, and 5× freeze/thaw cycles
Anti-Siglec-8 HEKA at 15 mg/mL with 0.030% PS80 at 1×, 3×, and 5× freeze/thaw cycles

The sample volume was 5.6 mL in an 8.0 mL HDPE bottle (70% fill). Slow freeze was conducted at −20° C. for at least 1 hour, then freeze completion at −80° C. for at least 1 hour. All samples were thawed at room temperature before batch-testing in the same run.

Analyses were performed using size exclusion chromatography (SEC), imaged capillary isoelectric focusing (icIEF), HIAC, and visual inspection/appearance. For SEC, the analysis was performed using a TSKgel G3000SWxl column (Tosoh #08541). The mobile phase was composed of 0.2M sodium phosphate pH 7.0. The sample was diluted to 5 mg/mL using the mobile phase as a diluent. Flow rate was 1.0 mL/min. Separation was performed at ambient temperature using an Agilent 1260 HPLC. Absorbance at 280 nm was monitored and used to determine peak area. icIEF was performed using an ICE3 instrument equipped with a fluorocarbon-coated capillary (FC) cartridge at ambient temperature. Additives used to assist separation and analysis were 4M urea, 4% pharmalytes covering 3-10 pH range, and low and high pI markers 4.65 and 9.22, respectively.

Agitation studies were performed at room temperature. Agitation speed of 10 rotations per minute were used in a rotary mixer. 20 test samples were prepared in 10R vials (7.0 mL fill):
Anti-Siglec-8 HEKA at 15 mg/mL with 0.0% PS80 at 0, 4, 8, 24, and 48 hours
Anti-Siglec-8 HEKA at 15 mg/mL with 0.015% PS80 at 0, 4, 8, 24, and 48 hours
Anti-Siglec-8 HEKA at 15 mg/mL with 0.025% PS80 at 0, 4, 8, 24, and 48 hours
Anti-Siglec-8 HEKA at 15 mg/mL with 0.030% PS80 at 0, 4, 8, 24, and 48 hours

As shown in Table J, the results of the freeze/thaw studies showed no significant changes in SoloVPE (14.8-14.9 mg/mL range). No significant trend and/or differences were observed by SEC up to 5× freeze/thaw cycles for 0.0%, 0.015%, 0.025%, and 0.030% PS 80 (99.2-99.3% monomer range). icIEF showed % acidic ≤1%, main ≤1.8%, and basic ≤1%. HIAC indicated that more particles were observed in the samples without PS80, as compared to with PS80. Antibody without PS80 at 1× freeze/thaw did not meet USP acceptance criteria for 10 μM (≤6000 counts/container). Antibody without PS80 at 3× and 5× freeze/thaw did not meet USP acceptance criteria for 10 μM (≤6000 counts/container) and 25 μM (≤600 counts/container). For appearance, samples without PS80 contained some visible particles.

As shown in Table K, the results of the agitation studies showed no changes in concentration by SoloVPE. By SEC, no significant trend and/or differences were observed up to 48 hours for 0.0%, 0.015%, 0.025%, and 0.030% PS 80 (99.2-99.1% monomer range). HIAC indicated that more particles were observed in the samples without PS80, as compared to with PS80. For appearance, samples without PS80 contained some visible particles.

Both freeze/thaw and agitation studies showed high particle counts by HIAC in samples without polysorbate 80. Freeze/thaw samples without polysorbate 80 failed USP acceptance criteria at 1×, 3×, and 5× for either ≥10 μM or ≥25 μM particle counts. This confirms the importance of polysorbate 80 in the formulations. All three polysorbate 80 concentrations in each study generated very similar analytical results, indicating little to no change in product quality after manipulation. This suggests that the concentration of PS80 in the formulations at 0.025% is a justifiable target, and the range tested (0.015%-0.030%) has generated similar acceptable results.

TABLE J
Freeze/thaw analytical results.

| | | SEC | | | icIEF | | | |
|---|---|---|---|---|---|---|---|---|
| Sample | soloVPE | % | % | % | % | % | % | pI of |
| Description | (mg/mL) | Aggregrates | Monomer | Fragments | Acidic | Main | Basic | main |
| HEKA IV | 14.9 | 0.7% | 99.3% | <0.1% | 20.9% | 66.7% | 12.4% | 8.33 |
| 15 mg/mL, | | | | (LOQ) | | | | |
| with 0.0% | | | | | | | | |
| PS80 0X | | | | | | | | |
| FT | | | | | | | | |
| HEKA IV | — | 0.7% | 99.3% | <0.1% | 20.7% | 66.8% | 12.4% | 8.34 |
| 15 mg/mL, | | | | (LOQ) | | | | |
| with 0.0% | | | | | | | | |
| PS80 1X | | | | | | | | |
| FT | | | | | | | | |
| HEKA IV | — | 0.7% | 99.3% | <0.1% | 21.0% | 66.9% | 12.1% | 8.34 |
| 15 mg/mL, | | | | (LOQ) | | | | |
| with 0.0% | | | | | | | | |
| PS80 3X | | | | | | | | |
| FT | | | | | | | | |
| HEKA IV | 14.9 | 0.7% | 99.3% | <0.1% | 20.7% | 66.7% | 12.6% | 8.32 |
| 15 mg/mL, | | | | (LOQ) | | | | |
| with 0.0% | | | | | | | | |
| PS80 5X | | | | | | | | |
| FT | | | | | | | | |
| HEKA IV | 14.9 | 0.8% | 99.2% | <0.1% | 20.9% | 66.7% | 12.4% | 8.34 |
| 15 mg/mL, | | | | (LOQ) | | | | |
| with | | | | | | | | |
| 0.015% | | | | | | | | |
| PS80 0X | | | | | | | | |
| FT | | | | | | | | |
| HEKA IV | — | 0.8% | 99.2% | <0.1% | 20.8% | 67.2% | 12.0% | 8.34 |
| 15 mg/mL, | | | | (LOQ) | | | | |
| with | | | | | | | | |
| 0.015% | | | | | | | | |
| PS80 1X | | | | | | | | |
| FT | | | | | | | | |
| HEKA IV | — | 0.8% | 99.2% | <0.1% | 20.4% | 67.5% | 12.0% | 8.35 |
| 15 mg/mL, | | | | (LOQ) | | | | |
| with | | | | | | | | |
| 0.015% | | | | | | | | |
| PS80 3X | | | | | | | | |
| FT | | | | | | | | |
| HEKA IV | 14.9 | 0.8% | 99.2% | <0.1% | 20.0% | 68.4% | 11.6% | 8.35 |
| 15 mg/mL, | | | | (LOQ) | | | | |
| with | | | | | | | | |
| 0.015% | | | | | | | | |
| PS80 5X | | | | | | | | |
| FT | | | | | | | | |
| HEKA IV | 14.9 | 0.8% | 99.2% | <0.1% | 20.8% | 67.1% | 12.1% | 8.34 |
| 15mg/mL, | | | | (LOQ) | | | | |
| with | | | | | | | | |
| 0.025% | | | | | | | | |
| PS80 0X | | | | | | | | |
| FT | | | | | | | | |
| HEKA IV | — | 0.8% | 99.2% | <0.1% | 20.7% | 66.6% | 12.6% | 8.32 |
| 15 mg/mL, | | | | (LOQ) | | | | |
| with | | | | | | | | |
| 0.025% | | | | | | | | |
| PS80 1X | | | | | | | | |
| FT | | | | | | | | |
| HEKA IV | — | 0.8% | 99.2% | <0.1% | 20.5% | 67.3% | 12.2% | 8.34 |
| 15 mg/mL, | | | | (LOQ) | | | | |
| with | | | | | | | | |
| 0.025% | | | | | | | | |
| PS80 3X | | | | | | | | |
| FT | | | | | | | | |
| HEKA IV | 14.9 | 0.8% | 99.2% | <0.1% | 20.8% | 66.9% | 12.4% | 8.33 |
| 15 mg/mL, | | | | (LOQ) | | | | |
| with | | | | | | | | |
| 0.025% | | | | | | | | |
| PS80 5X | | | | | | | | |
| FT | | | | | | | | |
| HEKA IV | 14.8 | 0.8% | 99.2% | <0.1% | 20.5% | 67.6% | 11.9% | 8.34 |
| 15 mg/mL, | | | | (LOQ) | | | | |
| with | | | | | | | | |
| 0.030% | | | | | | | | |
| PS80 0X | | | | | | | | |
| FT | | | | | | | | |
| HEKA IV | — | 0.8% | 99.2% | <0.1% | 20.4% | 67.3% | 12.4% | 8.32 |
| 15 mg/mL, | | | | (LOQ) | | | | |
| with | | | | | | | | |
| 0.030% | | | | | | | | |
| PS80 1X | | | | | | | | |
| FT | | | | | | | | |
| HEKA IV | — | 0.8% | 99.2% | <0.1% | 20.8% | 66.6% | 12.6% | 8.34 |
| 15 mg/mL, | | | | (LOQ) | | | | |
| with | | | | | | | | |
| 0.030% | | | | | | | | |
| PS80 3X | | | | | | | | |
| FT | | | | | | | | |
| HEKA IV | 14.8 | 0.8% | 99.2% | <0.1% | 20.7% | 66.9% | 12.5% | 8.34 |
| 15 mg/mL, | | | | (LOQ) | | | | |
| with | | | | | | | | |
| 0.030% | | | | | | | | |
| PS80 5X | | | | | | | | |
| FT | | | | | | | | |
| IAC control | — | 0.3% | 99.7% | <0.1% | 18.6% | 65.6% | 15.8% | 8.33 |
| | | | | (LOQ) | | | | |

| | HIAC (per container) | | | | | | |
|---|---|---|---|---|---|---|---|
| | ≥2 μm | ≥5 μm | | | Appearance | | |
| Sample | particles | particles | ≥10 μm | ≥25 μm | | | Visible |
| Description | (FIO) | (FIO) | ≤6000 | ≤600 | Color | Clarity | particles |
| HEKA IV | 254310 | 39568 | 5180 | 35 | <BY6 | <Ref III | CVP |
| 15 mg/mL, | | | | | | (18NTU) | |
| with 0.0% | | | | | | | |
| PS80 0X | | | | | | | |
| FT | | | | | | | |
| HEKA IV | 280735 | 78120 | 18060 | 315 | <BY6 | <Ref III | CVP |
| 15 mg/mL, | | | | | | (18NTU) | |
| with 0.0% | | | | | | | |
| PS80 1X | | | | | | | |
| FT | | | | | | | |
| HEKA IV | 454895 | 151358 | 28350 | 735 | =BY6 | =Ref III | CVP |
| 15 mg/mL, | | | | | | (18NTU) | |
| with 0.0% | | | | | | | |
| PS80 3X | | | | | | | |
| FT | | | | | | | |
| HEKA IV | 527293 | 199168 | 42578 | 648 | <BY5 | >Ref III | CVP |
| 15 mg/mL, | | | | | | (18NTU) | |
| with 0.0% | | | | | | | |
| PS80 5X | | | | | | | |
| FT | | | | | | | |
| HEKA IV | 4795 | 718 | 123 | 18 | <KBY6 | =Ref II | NVP |
| 15 mg/mL, | | | | | | (6NTU) | |
| with | | | | | | | |
| 0.015% | | | | | | | |
| PS80 0X | | | | | | | |
| FT | | | | | | | |
| HEKA IV | 5145 | 595 | 53 | 0 | =BY6 | =Ref II | NVP |
| 15 mg/mL, | | | | | | (6NTU) | |
| with | | | | | | | |
| 0.015% | | | | | | | |
| PS80 1X | | | | | | | |
| FT | | | | | | | |
| HEKA IV | 7350 | 683 | 70 | 0 | =BY6 | =Ref II | NVP |
| 15 mg/mL, | | | | | | (6NTU) | |
| with | | | | | | | |
| 0.015% | | | | | | | |
| PS80 3X | | | | | | | |
| FT | | | | | | | |
| HEKA IV | 4795 | 525 | 70 | 0 | =BY6 | =Ref II | NVP |
| 15 mg/mL, | | | | | | (6NTU) | |
| with | | | | | | | |
| 0.015% | | | | | | | |
| PS80 5X | | | | | | | |
| FT | | | | | | | |
| HEKA IV | 686 | 210 | 105 | 0 | <BY6 | <Ref II | NVP |
| 15 mg/mL, | | | | | | (6NTU) | |
| with | | | | | | | |
| 0.025% | | | | | | | |
| PS80 0X | | | | | | | |
| FT | | | | | | | |
| HEKA IV | 875 | 158 | 53 | 0 | <BY6 | =Ref II | NVP |
| 15mg/mL, | | | | | | (6NTU) | |
| with | | | | | | | |
| 0.025% | | | | | | | |
| PS80 1X | | | | | | | |
| FT | | | | | | | |
| HEKA IV | 1768 | 315 | 53 | 0 | =BY6 | =Ref II | NVP |
| 15 mg/mL, | | | | | | (6NTU) | |
| with | | | | | | | |
| 0.025% | | | | | | | |
| PS80 3X | | | | | | | |
| FT | | | | | | | |
| HEKA IV | 11953 | 1540 | 193 | 18 | =BY6 | =Ref II | NVP |
| 15 mg/mL, | | | | | | (6NTU) | |
| with | | | | | | | |
| 0.025% | | | | | | | |
| PS80 5X | | | | | | | |
| FT | | | | | | | |
| HEKA IV | 3115 | 508 | 123 | 0 | =BY6 | <Ref II | NVP |
| 15 mg/mL, | | | | | | (6NTU) | |
| with | | | | | | | |
| 0.030% | | | | | | | |
| PS80 0X | | | | | | | |
| FT | | | | | | | |
| HEKA IV | 1348 | 245 | 53 | 0 | =BY6 | =Ref II | NVP |
| 15 mg/mL, | | | | | | (6NTU) | |
| with | | | | | | | |
| 0.030% | | | | | | | |
| PS80 1X | | | | | | | |
| FT | | | | | | | |
| HEKA IV | 1575 | 368 | 70 | 0 | =BY6 | =Ref II | NVP |
| 15 mg/mL, | | | | | | (6NTU) | |
| with | | | | | | | |
| 0.030% | | | | | | | |
| PS80 3X | | | | | | | |
| FT | | | | | | | |
| HEKA IV | 2258 | 368 | 53 | 0 | =BY6 | =Ref II | NVP |
| 15 mg/mL, | | | | | | (6NTU) | |
| with | | | | | | | |
| 0.030% | | | | | | | |
| PS80 5X | | | | | | | |
| FT | | | | | | | |
| IAC control | | | | | | | |

TABLE K
Agitation analytical results.

| | | | | | HIAC (per container) | | | | | | |
|---|---|---|---|---|---|---|---|---|---|---|---|
| | | SEC | | | ≥2 μm | ≥5 μm | | | Appearance | | |
| Sample | SoloVPE | % | % | % | particles | particles | ≥10 μm | ≥25 μm | | | Visible |
| Description | (mg/mL) | Aggregates | Monomer | Fragments | (FIO) | (FIO) | ≤6000 | ≤600 | Color | Clarity | particles |
| HEKA IV | 14.6 | 0.8% | 99.2% | <0.1% | 26565 | 13335 | 2170 | 12 | ≤BY6 | =Ref | CVP |
| 15 mg/mL, | | | | (LOQ) | | | | | | II | |
| with 0.0% | | | | | | | | | | | |
| PS80 0 hrs | | | | | | | | | | | |
| HEKA IV | | 0.8% | 99.2% | <0.1% | 7128 | 1750 | 373 | 70 | ≤BY6 | =Ref | CVP |
| 15 mg/mL, | | | | (LOQ) | | | | | | II | |
| with 0.0% | | | | | | | | | | | |
| PS80 4 hrs | | | | | | | | | | | |
| HEKA IV | | 0.8% | 99.2% | <0.1% | 7957 | 2053 | 455 | 23 | ≤BY6 | =Ref | CVP |
| 15 mg/mL, | | | | (LOQ) | | | | | | II | |
| with 0.0% | | | | | | | | | | | |
| PS80 8 hrs | | | | | | | | | | | |
| HEKA IV | | 0.8% | 99.2% | <0.1% | 3570 | 957 | 152 | 12 | ≤BY6 | =Ref | CVP |
| 15 mg/mL, | | | | (LOQ) | | | | | | II | |
| with 0.0% | | | | | | | | | | | |
| PS80 24 hrs | | | | | | | | | | | |
| HEKA IV | 14.6 | 0.8% | 99.2% | <0.1% | 9112 | 2648 | 572 | 35 | ≤BY6 | =Ref | CVP |
| 15 mg/mL, | | | | (LOQ) | | | | | | II | |
| with 0.0% | | | | | | | | | | | |
| PS80 48 hrs | | | | | | | | | | | |
| HEKA IV | 14.6 | 0.9% | 99.1% | <0.1% | 653 | 175 | 70 | 0 | =BY6 | =Ref | NVP |
| 15 mg/mL, | | | | (LOQ) | | | | | | II | |
| with 0.015% | | | | | | | | | | | |
| PS80 0 hrs | | | | | | | | | | | |
| HEKA IV | | 0.9% | 99.1% | <0.1% | 210 | 23 | 12 | 12 | =BY6 | =Ref | NVP |
| 15 mg/mL, | | | | (LOQ) | | | | | | II | |
| with 0.015% | | | | | | | | | | | |
| PS80 4 hrs | | | | | | | | | | | |
| HEKA IV | | 0.9% | 99.1% | <0.1% | 175 | 12 | 0 | 0 | =BY6 | =Ref | NVP |
| 15 mg/mL, | | | | (LOQ) | | | | | | II | |
| with 0.015% | | | | | | | | | | | |
| PS80 8 hrs | | | | | | | | | | | |
| HEKA IV | | 0.9% | 99.1% | <0.1% | 268 | 152 | 70 | 0 | =BY6 | =Ref | NVP |
| 15 mg/mL, | | | | (LOQ) | | | | | | II | |
| with 0.015% | | | | | | | | | | | |
| PS80 24 hrs | | | | | | | | | | | |
| HEKA IV | 14.6 | 0.9% | 99.1% | <0.1% | 245 | 0 | 0 | 0 | =BY6 | =Ref | NVP |
| 15 mg/mL, | | | | (LOQ) | | | | | | II | |
| with 0.015% | | | | | | | | | | | |
| PS80 48 hrs | | | | | | | | | | | |
| HEKA IV | 14.6 | 0.9% | 99.1% | <0.1% | 327 | 93 | 23 | 0 | =BY6 | ≤Ref | NVP |
| 15 mg/mL, | | | | (LOQ) | | | | | | II | |
| with 0.025% | | | | | | | | | | | |
| PS80 0 hrs | | | | | | | | | | | |
| HEKA IV | | 0.9% | 99.1% | <0.1% | 222 | 70 | 23 | 12 | =BY6 | ≤Ref | NVP |
| 15 mg/mL, | | | | (LOQ) | | | | | | II | |
| with 0.025% | | | | | | | | | | | |
| PS80 4 hrs | | | | | | | | | | | |
| HEKA IV | | 0.9% | 99.1% | <0.1% | 303 | 117 | 23 | 0 | =BY6 | <Ref | NVP |
| 15 mg/mL, | | | | (LOQ) | | | | | | III | |
| with 0.025% | | | | | | | | | | | |
| PS80 8 hrs | | | | | | | | | | | |
| HEKA IV | | 0.9% | 99.1% | <0.1% | 292 | 93 | 47 | 12 | =BY6 | =Ref | NVP |
| 15 mg/mL, | | | | (LOQ) | | | | | | II | |
| with 0.025% | | | | | | | | | | | |
| PS80 24 hrs | | | | | | | | | | | |
| HEKA IV | 14.6 | 0.9% | 99.1% | <0.1% | 198 | 70 | 35 | 0 | =BY6 | =Ref | NVP |
| 15 mg/mL, | | | | (LOQ) | | | | | | II | |
| with 0.025% | | | | | | | | | | | |
| PS80 48 hrs | | | | | | | | | | | |
| HEKA IV | 14.6 | 0.9% | 99.1% | <0.1% | 187 | 35 | 12 | 0 | =BY6 | <Ref | NVP |
| 15 mg/mL, | | | | (LOQ) | | | | | | III | |
| with 0.030% | | | | | | | | | | | |
| PS80 0 hrs | | | | | | | | | | | |
| HEKA IV | | 0.9% | 99.1% | <0.1% | 385 | 70 | 12 | 0 | =BY6 | <Ref | NVP |
| 15 mg/mL, | | | | (LOQ) | | | | | | III | |
| with 0.030% | | | | | | | | | | | |
| PS80 4 hrs | | | | | | | | | | | |
| HEKA IV | | 0.9% | 99.1% | <0.1% | 35 | 12 | 12 | 0 | =BY6 | <Ref | NVP |
| 15 mg/mL, | | | | (LOQ) | | | | | | III | |
| with 0.030% | | | | | | | | | | | |
| PS80 8 hrs | | | | | | | | | | | |
| HEKA IV | | 0.9% | 99.1% | <0.1% | 128 | 23 | 0 | 0 | =BY6 | <Ref | NVP |
| 15 mg/mL, | | | | (LOQ) | | | | | | III | |
| with 0.030% | | | | | | | | | | | |
| PS80 24 hrs | | | | | | | | | | | |
| HEKA IV | 14.6 | 0.9% | 99.1% | <0.1% | 117 | 23 | 12 | 0 | =BY6 | <Ref | NVP |
| 15 mg/mL, | | | | (LOQ) | | | | | | III | |
| with 0.030% | | | | | | | | | | | |
| PS80 48 hrs | | | | | | | | | | | |
| IAC control | | 0.3% | 99.7% | <0.1% | | | | | | | |
| | | | | (LOQ) | | | | | | | |

SEQUENCES

All polypeptide sequences are presented N-terminal to C-terminal unless otherwise noted.
All nucleic acid sequences are presented 5′ to 3′ unless otherwise noted.
Amino acid sequence of 2E2 HVR-H1
IYGAH (SEQ ID NO: 1)
Amino acid sequence of 2E2 HVR-H2
VIWAGGSTNYNSALMS (SEQ ID NO: 2)
Amino acid sequence of 2E2 HVR-H3
DGSSPYYYSMEY (SEQ ID NO: 3)
Amino acid sequence of 2E2 HVR-L1
SATSSVSYMH (SEQ ID NO: 4)
Amino acid sequence of 2E2 HVR-L2
STSNLAS (SEQ ID NO: 5)
Amino acid sequence of 2E2 HVR-L3
QQRSSYPFT (SEQ ID NO: 6)
Amino acid sequence of 2E2 RHE heavy chain variable domain
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYSMEYWGQGT
TVTVSS (SEQ ID NO: 7)
Amino acid sequence of 2E2 RKA light chain variable domain
EIVLTQSPATLSLSPGERATLSCSATSSVSYMHWFQQKPGQAPRLLIYSTSNLASGIPARF
SGSGSGTDFTLTISSLEPEDFAVYYCQQRSSYPFTFGPGTKLDIK (SEQ ID NO: 8)
Amino acid sequence of 2E2 RKF light chain variable domain
EIVLTQSPATLSLSPGERATLSCSATSSVSYMHWFQQKPGQAPRLLIYSTSNLASGIPARF
SGSGSGTDYTLTISSLEPEDFAVYYCQQRSSYPFTFGPGTKLDIK (SEQ ID NO: 9)
Amino acid sequence of exemplary heavy chain FR1
EVQLVESGGGLVQPGGSLRLSCAASGFSLT (SEQ ID NO: 10)
Amino acid sequence of exemplary heavy chain FR2
WVRQAPGKGLEWVG (SEQ ID NO: 11)
Amino acid sequence of exemplary heavy chain FR3
RFTISKDNSKNTVYLQMNSLRAEDTAVYYCAR (SEQ ID NO: 12)
Amino acid sequence of exemplary heavy chain FR4
WGQGTTVTVSS (SEQ ID NO: 13)
Amino acid sequence of exemplary light chain FR1
EIVLTQSPATLSLSPGERATLSC (SEQ ID NO: 14)
Amino acid sequence of exemplary light chain FR2
WFQQKPGQAPRLLIY (SEQ ID NO: 15)
Amino acid sequence of exemplary light chain FR3
GIPARFSGSGSGTDFTLTISSLEPEDFAVYYC (SEQ ID NO: 16)
Amino acid sequence of exemplary light chain FR3
GIPARFSGSGSGTDYTLTISSLEPEDFAVYYC (SEQ ID NO: 17)
Amino acid sequence of exemplary light chain FR4
FGPGTKLDIK (SEQ ID NO: 18)
Amino acid sequence of HEKA heavy chain and HEKF heavy chain
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYSMEYWGQGT
TVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG (SEQ ID NO: 19)
Amino acid sequence of HEKA light chain
EIVLTQSPATLSLSPGERATLSCSATSSVSYMHWFQQKPGQAPRLLIYSTSNLASGIPARF
SGSGSGTDFTLTISSLEPEDFAVYYCQQRSSYPFTFGPGTKLDIKRTVAAPSVFIFPPSDEQ
LKSGTASVVCLLNNFYPREAKVQWKVDNALQSGNSQESVTEQDSKDSTYSLSSTLTLS
KADYEKHKVYACEVTHQGLSSPVTKSFNRGEC (SEQ ID NO: 20)
Amino acid sequence of HEKF light chain
EIVLTQSPATLSLSPGERATLSCSATSSVSYMHWFQQKPGQAPRLLIYSTSNLASGIPARF
SGSGSGTDYTLTISSLEPEDFAVYYCQQRSSYPFTFGPGTKLDIKRTVAAPSVFIFPPSDEQ
LKSGTASVVCLLNNFYPREAKVQWKVDNALQSGNSQESVTEQDSKDSTYSLSSTLTLS
KADYEKHKVYACEVTHQGLSSPVTKSFNRGEC (SEQ ID NO: 21)
Amino acid sequence of IgG1 heavy chain constant region
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVD
KSRWQQGNVFSCSVMHEALHNHYTQKSLSLSPG (SEQ ID NO: 22)
Amino acid sequence of IgG4 heavy chain constant region
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLG (SEQ ID NO: 23)
Amino acid sequence of Ig kappa light chain constant region
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC (SEQ ID NO: 24)
Human Siglec-8 Amino Acid Sequence
GYLLQVQELVTVQEGLCVHVPCSFSYPQDGWTDSDPVHGYWFRAGDRPYQDAPVATN
NPDREVQAETQGRFQLLGDIWSNDCSLSIRDARKRDKGSYFFRLERGSMKWSYKSQLN
YKTKQLSVFVTALTHRPDILILGTLESGHSRNLTCSVPWACKQGTPPMISWIGASVSSPG
PTTARSSVLTLTPKPQDHGTSLTCQVTLPGTGVTTTSTVRLDVSYPPWNLTMTVFQGDA
TASTALGNGSSLSVLEGQSLRLVCAVNSNPPARLSWTRGSLTLCPSRSSNPGLLELPRVH
VRDEGEFTCRAQNAQGSQHISLSLSLQNEGTGTSRPVSQVTLAAVGGAGATALAFLSFC
IIFIIVRSCRKKSARPAAGVGDTGMEDAKAIRGSASQGPLTESWKDGNPLKKPPPAVAPS
SGEEGELHYATLSFHKVKPQDPQGQEATDSEYSEIKIHKRETAETQACLRNHNPSSKEV
RG (SEQ ID NO: 25)
Human Siglec-8 Amino Acid Sequence
GYLLQVQELVTVQEGLCVHVPCSFSYPQDGWTDSDPVHGYWFRAGDRPYQDAPVATN
NPDREVQAETQGRFQLLGDIWSNDCSLSIRDARKRDKGSYFFRLERGSMKWSYKSQLN
YKTKQLSVFVTALTHRPDILILGTLESGHPRNLTCSVPWACKQGTPPMISWIGASVSSPG
PTTARSSVLTLTPKPQDHGTSLTCQVTLPGTGVTTTSTVRLDVSYPPWNLTMTVFQGDA
TASTALGNGSSLSVLEGQSLRLVCAVNSNPPARLSWTRGSLTLCPSRSSNPGLLELPRVH
VRDEGEFTCRAQNAQGSQHISLSLSLQNEGTGTSRPVSQVTLAAVGGAGATALAFLSFC
IIFIIVRSCRKKSARPAAGVGDTGMEDAKAIRGSASQGPLTESWKDGNPLKKPPPAVAPS
SGEEGELHYATLSFHKVKPQDPQGQEATDSEYSEIKIHKRETAETQACLRNHNPSSKEV
RG (SEQ ID NO: 26)
Amino acid sequence of HEKA IgG4 heavy chain (IgG4 contains a S228P mutation)
EVQLVESGGGLVQPGGSLRLSCAASGFSLTIYGAHWVRQAPGKGLEWVGVIWAGGST
NYNSALMSRFTISKDNSKNTVYLQMNSLRAEDTAVYYCARDGSSPYYYSMEYWGQGT
TVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRL
TVDKSRWQEGNVFSCSVMHEALHNHYTQKSLSLSLG (SEQ ID NO: 27)

1. A liquid formulation comprising (a) a monoclonal antibody that binds to a human Siglec-8 in a concentration of about 5 mg/mL to about 15 mg/mL; (b) arginine in a concentration of about 50 mM to about 200 mM; (c) succinate in a concentration of about 5 mM to about 50 mM; (d) sodium chloride in a concentration of about 40 mM to about 150 mM; and (e) polysorbate in a concentration of about 0.002% to about 0.05%;
wherein the antibody comprises: (1) a heavy chain variable region comprising: an HVR-H1 comprising the amino acid sequence of SEQ ID NO:1; an HVR-H2 comprising the amino acid sequence of SEQ ID NO:2; an HVR-H3 comprising the amino acid sequence of SEQ ID NO:3; and (1) a light chain variable region comprising: an HVR-L1 comprising the amino acid sequence of SEQ ID NO:4; an HVR-L2 comprising the amino acid sequence of SEQ ID NO:5; and an HVR-L3 comprising the amino acid sequence of SEQ ID NO:6.

2. The formulation of claim 1, wherein the antibody is in a concentration of about 15 mg/mL.

3. The formulation of claim 1, comprising arginine in a concentration of about 100 mM to about 200 mM.

4-5. (canceled)

6. The formulation of claim 1, wherein the arginine is an arginine HCl salt.

7. The formulation of claim 1, comprising succinate in a concentration of about 10 mM to about 50 mM.

8-9. (canceled)

10. The formulation of claim 1, wherein the succinate is a sodium succinate salt.

11. The formulation of claim 1, comprising sodium chloride in a concentration of about 50 mM to about 130 mM.

12-13. (canceled)

14. The formulation of claim 1, comprising polysorbate in a concentration of about 0.01% to about 0.05%.

15. (canceled)

16. The formulation of claim 1, wherein the polysorbate is polysorbate-80.

17. The formulation of claim 1, wherein the formulation has a pH of about 5.0 to about 7.0.

18. (canceled)

19. The formulation of claim 1, comprising (a) the antibody in a concentration of 15 mg/mL; (b) arginine in a concentration of 125 mM; (c) succinate in a concentration of 20 mM; (d) sodium chloride in a concentration of 80 mM; and (e) polysorbate in a concentration of 0.025%, wherein the pH of the formulation is 6.0.

20-21. (canceled)

22. The formulation of claim 1, wherein:
(a) less than 5% of the antibody in the formulation is aggregated after shaking overnight at 800 rpm, as measured by abundance of small antibody oligomers by size-exclusion chromatography high-performance liquid chromatography (SEC-HPLC);
(b) after freezing and thawing the formulation has an absorbance at 400 nm (A400 nm) of less than about 150% of A400 nm of a reference standard, as measured by UV-Vis spectroscopy; and/or
(c) the formulation has an absorbance at 400 nm (A400 nm) of less than about 0.1 after shaking overnight at 800 rpm, as measured by UV-Vis spectroscopy.

23-25. (canceled)

26. The formulation of claim 1, wherein the antibody comprises a Fc region and N-glycoside-linked carbohydrate chains linked to the Fc region, wherein less than 50% of the N-glycoside-linked carbohydrate chains of the antibody in the formulation contain a fucose residue.

27. (canceled)

28. The formulation of claim 1, wherein the antibody comprises a heavy chain variable region comprising the amino acid sequence of SEQ ID NO:7 and a light chain variable region comprising the amino acid sequence of SEQ ID NO:8 or 9.

29. The formulation of claim 28, wherein the antibody comprises a heavy chain Fc region comprising a human IgG Fc region.

30. The formulation of claim 29, wherein the human IgG Fc region comprises a human IgG1 Fc region or a human IgG4 Fc region.

31. The formulation of claim 30, wherein the human IgG1 Fc region is non-fucosylated.

32. (canceled)

33. The formulation of claim 30, wherein the human IgG4 Fc region comprises the amino acid substitution S228P, wherein the amino acid residues are numbered according to the EU index as in Kabat.

34-35. (canceled)

36. The formulation of claim 29, wherein at least one or two of the heavy chains of the antibody is non-fucosylated.

37. The formulation of claim 1, wherein the antibody comprises a heavy chain comprising the amino acid sequence of SEQ ID NO:19 and a light chain comprising the amino acid sequence of SEQ ID NO:20 or 21.

38-42. (canceled)
